(19) United States
(12) Patent Application Publication
(10) Pub. No.: US 2022/0098627 A1
(43) Pub. Date: Mar. 31, 2022

(54) A process for the bioproduction of glycolate

(71) Applicant: Photanol B.V., Amsterdam (NL)

(72) Inventors: Aniek Doreen van der Woude, Amstelveen (NL); Klaas Jan Hellingwerf, Amsterdam (NL); Koen Mulder, Amsterdam (NL)

(73) Assignee: Photanol B.V., Amsterdam (NL)

(21) Appl. No.: 17/424,174

(22) PCT Filed: Jan. 24, 2020
(86) PCT No.: PCT/EP2020/051785

§ 371 Date: Jul. 20, 2021

(30) Foreign Application Priority Data
Jan. 24, 2019 (EP) 19153544.2

(51) Int. Cl. C12P 7/42; C12N 9/88; C12N 9/16; C12N 1/18; C12N 1/20

(52) U.S. Cl. CPC C12P 7/42; C12N 9/88; C12N 9/16; C12R 2001/19; C12N 1/205; C12Y 401/01039; C12Y 301/03018; C12N 1/185

(57) ABSTRACT

The present invention relates to the field of biochemistry, specifically to the bioproduction of glycolate. Host cells, especially cyanobacteria of the genus Synechocystis, are modified in several ways to increase extracellular glycolate, including: mutant Rubisco enzymes, overexpression of phosphoribulokinase (PRK) or phosphoglycolate phosphatase (PGP), a permease to export glycolate, like GIcA, or by reduction of the capacity to metabolize glycolate due to reduced or eliminated glycolate dehydrogenase, glycolate oxidase activity and/or lactate dehydrogenase.

### FIELD OF THE INVENTION

The present invention relates to the field of biochemistry, specifically to the bioproduction of glycolate.

### BACKGROUND OF THE INVENTION

Glycolate, the conjugate base of glycolic acid, is the simplest α-hydroxy acid with the gross brute formula C₂H₄O₃. It has multiple applications, primarily as a skin/personal care agent, but also in the textile industry as a dyeing and tanning agent and in food processing as a flavoring agent and as a preservative. It is also used in adhesives and plastics and is often included into emulsion polymers, solvents and additives for ink and paint, in order to improve flow properties and impart gloss. Glycolate can be produced either via chemical synthesis or via microbial fermentation. Currently, most of the glycolate is chemically manufactured by high-pressure, high-temperature carbonylation of formaldehyde (Loder, 1939). Glycolate can also be produced through bioconversion of glycolonitrile using microbial nitrilases (He et al., 2010) or bioconversion of ethylene glycol to glycolate by bacteria such as Gluconobacter oxydans (Wei et al., 2009). WO2013050659 relates to the production of glycolic acid in eukaryotic cells, including yeast cells and filamentous fungi, genetically modified to express a glyoxylate reductase gene to produce glycolic acid. WO2016193540 relates to the production of glycolic acid in eukaryotic cells wherein the entire glycolic acid production pathway is introduced into the cytosol. EP2233562 relates to the production of glycolic acid in E. coli. WO2011036213 relates to the production of glycolic acid in bacteria and yeast wherein the pH is first lower than 7 and subsequently is higher than 7. WO2007140816 relates to the production of glycolate in E. coli transformed i) to attenuate the glyoxylate consuming pathways to other compounds than glycolate ii) to use an NADPH glyoxylate reductase to convert glyoxylate to glycolate iii) to attenuate the level of all the glycolate metabolizing enzymes and iv) increase the flux in the glyoxylate/glycolate pathway. WO2017059236 relates to the production of glycolate by fermentation of pentose sugars like xylulose and ribulose.

However, for production of glycolate using chemotrophic microorganisms, substrates such as glucose are needed, which makes the production process economically, and with respect to sustainability, rather inefficient. Taubert et al., 2019 have reported production of glycolate in the unicellular algae Chlamydomonas using CO₂ as carbon source under modulated culture conditions. Cyanobacteria have been reported for the production of metabolites and organic acids.

WO2009078712 relates to the production of various compounds in cyanobacteria, such as butanol, ethanol, ethylene, succinate, propanol, acetone and D-lactate. WO2011136639 relates to the production of L-lactate in cyanobacteria. WO2014092562 relates to the production of acetoin, 2,3-butanediol and 2-butanol in cyanobacteria. WO2015147644 relates to the production of erythritol in cyanobacteria. WO2016008883 relates to the production of various monoterpenes in cyanobacteria. WO2016008885 relates to the production of various sesquiterpenes in cyanobacteria. Eisenhut et al. (2008) relate to the CO₂ concentrating mechanism of cyanobacteria. A Synechocystis mutant overexpressing the putative phosphoglycolate phosphatases slr0458 was constructed. Compared with the wild type, the mutant grew slower under limiting CO₂ concentration and the intracellular 2-phosphoglycolate level was considerably smaller than in the wild type Synechocystis. Haimovich-Dayan et al, 2014 investigates the photorespiratory 2-phosphoglycolate (2PG) metabolism in Synechocystis PCC6803; it is demonstrated that a mutant defective in its two glycolate dehydrogenases (ΔglcD1/ΔglcD2) was unable to grow under low CO₂ conditions. Pierce et al, 1989 demonstrates that the native ribulose bisphosphate carboxylase (Rubisco) is essential for both photoautotrophic growth and photoheterotrophic growth of the cyanobacterium Synechocystis PCC6803. By exchanging the native Rubisco for a heterologous one (from Rhodospirillum rubrum) with a lower affinity for CO₂, a mutant was obtained that was extremely sensitive to the CO₂/O₂ ratio supplied during growth and was unable to grow at all in air. As depicted here above, one has succeeded in producing various compounds in cyanobacteria; however, the yields vary between products and for some products the yield appears still too low to be commercially relevant.

While cyanobacteria natively produce some glycolate, there is no disclosure nor suggestion of producing glycolate on a commercially relevant scale. At present, there is thus no efficient bioprocess for the production of glycolate available, neither on laboratory scale, nor on industrial scale, while using CO₂ as the substrate. Thus, in view of the state of the art, there is still a need for an alternative, more sustainable and improved glycolate production process, without the need for expensive and complicated starting materials, and with a commercially relevant yield.

### BRIEF DESCRIPTION OF THE FIGURES

FIG. 1. Metabolic pathways for glycolic acid production with possible genetic modifications. (1) deletion of glycolate dehydrogenase(s)/oxidase(s) (glcD1/2), (2) overexpression of phosphoglycolate phosphatase (PGP), (3) Rubisco with increased affinity for 02 and higher turnover activity, optionally together with deletion of endogenous Rubisco, and/or overexpression of phoshoribulokinase (PRK), (4) overexpression of a permease (glcA), (5) overexpression of glyoxylate reductase (GlyR), (6) overexpression of isocitrate lyase (aceA).

FIG. 2. Growth (filled symbols) and glycolate production (open symbols) of the following Synechocystis strains: (A) wildtype, SGP009m (ΔglcD1+ΔglcD2), SGP026 (ΔglcD1+ΔglcD2+slr0168::Ptrc_coPGPCr), and SGP038 (ΔglcD1+ΔglcD2+slr0168::PspBA2 coGIcAEc); (B) SGP171 (wildtype with empty pAVO+), SGP172 (SGP009m with empty pAVO+), SGP173 (wildtype with +pAVO+_ptrc1_GlyR1At), and SGP174 (SGP009m+pAVO+_ptrc1_GlyR1At).

FIG. 3. Growth (filled symbols) and glycolate production (open symbols) of the following Synechocystis strains: SGP201 (ΔglcD1+ΔglcD2+Δldh+slr0168::Ptrc1 pgpCr) and SGP237 (ΔglcD1+ΔglcD2+Δldh+slr0168::Ptrc1 pgpCr+ΔrcbLXS::PcpcBA_rbcMRr).

FIG. 4. Growth (filled symbols) and glycolate production (open symbols) of the following Synechocystis strains: SAW082 (ΔglcD1+ΔglcD2+Δldh) and SGP214 (ΔglcD1+ΔglcD2+Δldh+pAVO+_ptrc1_AceAEc_GlyR1At).

FIG. 5. Growth (filled symbols) and glycolate production (open symbols) of the following Synechocystis strains: (A) SGP237m (ΔglcD1+ΔglcD2+Δldh+slr0168::Ptrc1 pgpCr+ΔrcbLXS::PcpcBA_rbcMRr) and SGP338 (ΔglcD1+ΔglcD2+Δldh+slr0168::kanR+ΔrcbLXS::PcpcBA_rbcMRr); (B) SGP340 (ΔglcD1+ΔglcD2+Δldh+slr0168::Ptrc1 pgpCr+ΔrcbLXS::PcpcBA_rbcMRs) and SGP341 (ΔglcD1+ΔglcD2+Δldh+slr0168::Ptrc1 pgpCr+ΔrcbLXS::PcpcBA rbcMRc); (C) SGP371 (ΔglcD1+ΔglcD2+Δldh+slr0168::Ptrc1 pgpCr+ΔrcbLXS::PcpcBA_rbcMRr+NSC2::Ptrc_rbcLXS).

FIG. 6. Growth (filled symbols) and glycolate production (open symbols) of the following strains: (A) Synechococcus PCC7002 strain ScGP006 (ΔglcD1::kanR+ΔrcbLXS::PcpcBA_rbcMRr-camR); (B) Synechococcus elongatus PCC7942 strain SeGP004 (ΔglcD1::Ptrc1 pgpCr-kanR+ΔrcbLXS::PcpcBA_rbcMRr-camR).

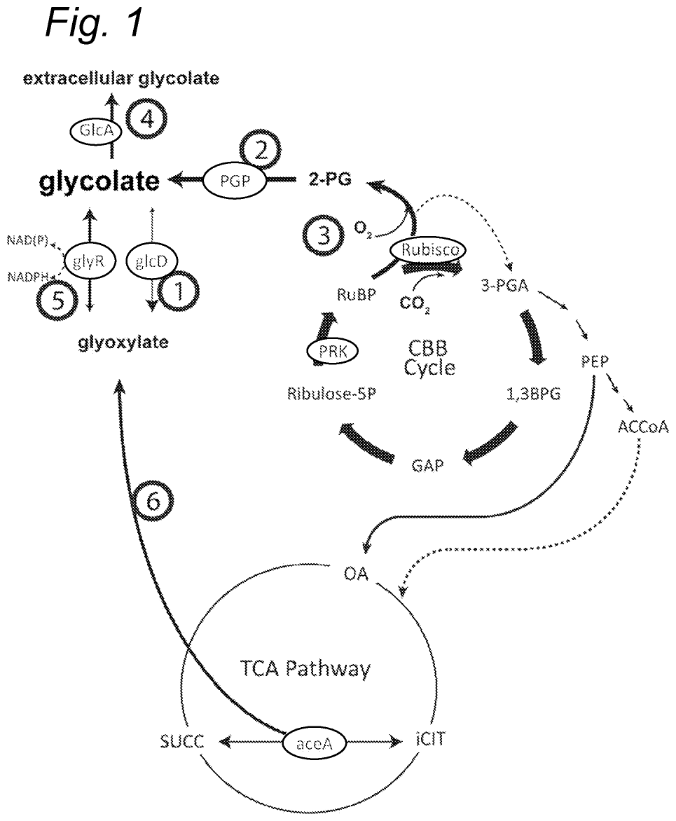

### OVERVIEW OF SEQUENCES

| SEQ ID NO | Name | Organism |
|---|---|---|
| 1 | Glycolate dehydrogenase 1 (sll0404) CDS | Synechocystis PCC6803 |
| 2 | Glycolate dehydrogenase 1 PRT | Synechocystis PCC6803 |
| 3 | Glycolate dehydrogenase 2 (slr0806) CDS | Synechocystis PCC6803 |
| 4 | Glycolate dehydrogenase 2 PRT | Synechocystis PCC6803 |
| 5 | Lactate dehydrogenase (slr1556) CDS | Synechocystis PCC6803 |
| 6 | Lactate dehydrogenase PRT | Synechocystis PCC6803 |
| 7 | Phosphoglycolate phosphatase (pgpEc) CDS | Escherichia coli |
| 8 | Phosphoglycolate phosphatase PRT | Escherichia coli |
| 9 | Phosphoglycolate phosphatase (pgpCr) CDS | Chlamydomonas |
| | | reinhardtii |
| 10 | Phosphoglycolate phosphatase PRT | Chlamydomonas |
| | | reinhardtii |
| 11 | Phosphoglycolate phosphatase (pgpSyn7942) CDS | Synechococcus |
| | | elongatus PCC7942 |
| 12 | Phosphoglycolate phosphatase PRT | Synechococcus |
| | | elongatus PCC7942 |
| 13 | Glycolate permease (glcA) CDS | Escherichia coli |
| 14 | Glycolate permease PRT | Escherichia coli |
| 15 | Rubisco (RbcMRr) CDS | Rhodospirillum rubrum |
| 16 | Rubisco PRT | Rhodospirillum rubrum |
| 17 | Rubisco (RbcMH44N) CDS | Rhodospirillum rubrum |
| 18 | Rubisco PRT | Rhodospirillum rubrum |
| 19 | Rubisco CDS | Archaeolobus fulgidus |
| 20 | Rubisco PRT | Archaeolobus fulgidus |
| 21 | Rubisco operon (rbcLXS) CDS | Synechocystis PCC6803 |
| 22 | Rubisco PRT | Synechocystis PCC6803 |
| 23 | Rubisco (RbcX) PRT | Synechocystis PCC6803 |
| 24 | Rubisco (RbcS) PRT | Synechocystis PCC6803 |
| 25 | Isocitrate lyase (aceAEc) CDS | Escherichia coli |
| 26 | Isocitrate lyase PRT | Escherichia coli |
| 27 | Isocitrate lyase (aceAMt) CDS | Mycobacterium |
| | | tuberculosis |
| 28 | Isocitrate lyase PRT | Mycobacterium |
| | | tuberculosis |
| 29 | Glyoxylate reductase (GlyR1At) CDS | Arabidopsis thaliana |
| 30 | Glyoxylate reductase PRT | Arabidopsis thaliana |
| 31 | Phosphoribulokinase (prk) CDS | Synechococcus |
| | | elongatus PCC 7942 |
| 32 | Phosphoribulokinase PRT | Synechococcus |
| | | elongatus PCC 7942 |
| 33 | PBRS-mazF cassette polynucleotide | Artificial sequence |
| 34 | ccmM (sll1031) | Synechocystis PCC6803 |
| 35 | pHKH-RFP polynucleotide | Artificial sequence |
| 36 | pAVO+ (RSF1010) | Artificial sequence |
| 37 | Ptrc1 | Artificial sequence |
| 38 | PcpcBA | Artificial sequence |
| 39 | PpsbA2 | Artificial sequence |
| 40 | PrbcL | Artificial sequence |
| 41 | Hom1sll0404_F | Artificial sequence |
| 42 | Hom1sll0404_R | Artificial sequence |
| 43 | Hom2sll0404_F | Artificial sequence |
| 44 | Hom2sll0404_R | Artificial sequence |
| 45 | Hom1slr0806_F | Artificial sequence |
| 46 | Hom1slr0806_R | Artificial sequence |
| 47 | Hom2slr0806_F | Artificial sequence |
| 48 | Hom2slr0806_R | Artificial sequence |
| 49 | slr0806_IN_F | Artificial sequence |
| 50 | slr0806_IN_R | Artificial sequence |
| 51 | sll0404_IN_F | Artificial sequence |
| 52 | sll0404_IN_R | Artificial sequence |
| 53 | Ndel_pgp_Syn_F | Artificial sequence |
| 54 | BamHI_pgp_Syn_R | Artificial sequence |
| 55 | Slr1556-HOM1-F | Artificial sequence |
| 56 | Slr1556-HOM1-R | Artificial sequence |
| 57 | Slr1556-HOM2-F | Artificial sequence |
| 58 | Slr1556-HOM2-R | Artificial sequence |
| 59 | slr1556_F | Artificial sequence |
| 60 | slr1556_R | Artificial sequence |
| 61 | Nhel_RBS_Ndel_aceA | Artificial sequence |
| 62 | aceA_BamHI_AvrII | Artificial sequence |
| 63 | Ndel-rbcL-7942F | Artificial sequence |
| 64 | BamHI-rbcS-7942R | Artificial sequence |
| 65 | Rbc-HR1-F | Artificial sequence |
| 66 | Rbc-HR1-R | Artificial sequence |
| 67 | Rbc-HR2-F | Artificial sequence |
| 68 | Rbc-HR2-R | Artificial sequence |
| 69 | RbcM_Rr_Ndel_F | Artificial sequence |
| 70 | RbcM_Rr_Spel_R | Artificial sequence |
| 71 | RbcM_H44N_F | Artificial sequence |
| 72 | RbcM_H44N_R | Artificial sequence |
| 73 | RbcX-5UTR-Nhel-F | Artificial sequence |
| 74 | Xbal-cpcBA-F | Artificial sequence |
| 75 | RbcX-BglII-F | Artificial sequence |
| 76 | Hom1_sll1031_F | Artificial sequence |
| 77 | Hom1_sll1031_R | Artificial sequence |
| 78 | Hom2_sll1031_F | Artificial sequence |
| 79 | Hom2_sll1031_R | Artificial sequence |
| 80 | RbcL_F140I_F | Artificial sequence |
| 81 | RbcL_F140I_R | Artificial sequence |
| 82 | RbcL_F345I_F | Artificial sequence |
| 83 | RbcL_F345I_R | Artificial sequence |
| 84 | Rubisco operon (rbcLS) CDS | Synechococcus |
| | | elongatus PCC 7942 |
| 85 | Rubisco (RbcL) PRT | Synechococcus |
| | | elongatus PCC 7942 |
| 86 | Rubisco (RbcL_{F140I}) PRT | Synechococcus |
| | | elongatus PCC 7942 |
| 87 | Rubisco (RbcL_{F345I}) PRT | Synechococcus |
| | | elongatus PCC 7943 |
| 88 | Rubisco (RbcL_{F140I/F345I}) PRT | Synechococcus |
| | | elongatus PCC 7944 |
| 89 | Rubisco (RbcS) PRT | Synechococcus |
| | | elongatus PCC 7942 |
| 90 | Rubisco CDS | Rhodopseudomonas |
| | | capsulatus |
| 91 | Rubisco PRT | Rhodopseudomonas |
| | | capsulatus |
| 92 | Rubisco CDS | Rhodobacter |
| | | sphaeroides |
| 93 | Rubisco PRT | Rhodobacter |
| | | sphaeroides |
| 94 | Pcpt | Synechocystis PCC6803 |
| 95 | Glycolate dehydrogenase 1 (A2859) CDS | Synechococcus PCC7002 |
| 96 | Glycolate dehydrogenase 1 PRT | Synechococcus PCC7002 |
| 97 | Rubisco operon (rbcLXS) CDS | Synechococcus PCC7002 |
| 98 | Rubisco (RbcL) PRT | Synechococcus PCC7002 |
| 99 | Rubisco (RbcX) PRT | Synechococcus PCC7002 |
| 100 | Rubisco (RbcS) PRT | Synechococcus PCC7002 |
| 101 | Glycolate dehydrogenase 1 CDS | Synechococcus |
| | | elongatus PCC 7942 |
| 102 | Glycolate dehydrogenase 1 PRT | Synechococcus |
| | | elongatus PCC 7942 |
| 103 | RbcRs_Ndel_F | Artificial sequence |
| 104 | RbcRs_Spel_R | Artificial sequence |
| 105 | RbcRp_Ndel_F | Artificial sequence |
| 106 | RbcRp_Spel_R | Artificial sequence |
| 107 | 7002glcD1-HOM1-f | Artificial sequence |
| 108 | 7002glcD1-HOM1overlap-R | Artificial sequence |
| 109 | 7002glcD1-HOM2overlap-F | Artificial sequence |
| 110 | 7002glcD1-HOM2-R | Artificial sequence |
| 111 | UTEXglcD1-HOM1-f | Artificial sequence |
| 112 | UTEXglcD1-HOM1overlap-R | Artificial sequence |
| 113 | UTEXglcD1-HOM2overlap-F | Artificial sequence |
| 114 | UTEXglcD1-HOM2-R | Artificial sequence |
| 115 | Rbc7002_HR1_F | Artificial sequence |
| 116 | Rbc7002_HR1_R | Artificial sequence |
| 117 | Rbc7002_HR2_F | Artificial sequence |
| 118 | Rbc7002_HR2_R | Artificial sequence |
| 119 | Rbc7942_HR1_F | Artificial sequence |
| 120 | Rbc7942_HR1_R | Artificial sequence |
| 121 | Rbc7942_HR2_F | Artificial sequence |
| 122 | Rbc7942_HR2_R | Artificial sequence |

CDS: coding sequence;
PRT: protein sequence;
_F: forward primer;
_R: reverse primer

### DESCRIPTION OF THE INVENTION

The inventors have arrived at an improved process for the production of extracellular glycolate with a commercially relevant yield.

Accordingly, the invention provides for a recombinant host cell for the production of extracellular glycolate, wherein the host cell:
is derived from a parent host cell,
comprises phosphoribulokinase (PRK) and ribulose bisphosphate carboxylase (Rubisco) activity,
is substantially unable to anabolize glycolate, optionally,
comprises increased phosphoglycolate phosphatase activity compared to the parent host cell, and optionally,
comprises a permease to export glycolate out of the host cell into the culture medium.

The production of extracellular glycolate is herein to be construed in such a way that the glycolate produced in the host cell is secreted, whether actively or passively, by the host cell, e.g. mediated by a transporter and/or a permease, and/or via non-facilitated diffusion across the cyanobacterial cell envelope. Leakage of the glycolate by lysis of host cells is preferably not within the scope of the invention.

Substantially unable to anabolize glycolate is herein to be construed that less than about 10% of the glycolate produced is anabolized by the host cell. In an embodiment, less than about 9%, 8%, 7%, 6%, 5%, 4%, 3%, 2% or less than 1% of glycolate produced is anabolized by the host cell. In the recombinant host cell for the production of extracellular glycolate, the ribulose 1,5-bisphosphate carboxylase/oxygenase (Rubisco) activity may be with increased selectivity for 02 compared to the Rubisco of the parent cell. The selectivity may be increased by at least 10%, 20%, 30%, 40%, 50%, 60%, 70%, 80%, 90%, or at least 100%. The selectivity may be increased by at least 1-fold, 2-fold, 3-fold, 4-fold, 5-fold, 6-fold, 7-fold, 8-fold, 9-fold, 10-fold, 1 log, 2 log, 3 log, or at least 4 log.

In the recombinant host cell for the production of extracellular glycolate, the host cell may be substantially unable to metabolize glycolate due to reduced or eliminated glycolate dehydrogenase, glycolate oxidase activity and/or lactate dehydrogenase activity relative to the parent cell. The glycolate dehydrogenase activity may be reduced by at least 10%, 20%, 30%, 40%, 50%, 60%, 70%, 80%, 90%, 95%, 96%, 97%, 98%, 99% or may be reduced completely (elimination). The lactate dehydrogenase activity may be reduced by at least 10%, 20%, 30%, 40%, 50%, 60%, 70%, 80%, 90%, 95%, 96%, 97%, 98%, 99% or may be reduced completely (elimination). The person skilled in the art knows how to reduce activity of an enzyme, e.g. by reduction of expression of the sequence encoding the enzyme by gene disruption (knock-out) or down regulation.

Accordingly, in the recombinant host cell for the production of extracellular glycolate, the reduced or eliminated glycolate dehydrogenase and/or glycolate oxidase activity relative to the parent cell may be due to targeted gene disruption of deletion of a glycolate dehydrogenase and/or glycolate oxidase and/or lactate dehydrogenase. Preferred glycolate dehydrogenase, glycolate oxidase and lactate dehydrogenase are the ones described elsewhere herein.

In the recombinant host cell for the production of extracellular glycolate, the host cell may overexpress glyoxylate reductase and/or isocitrate lyase in view of the parent cell. Overexpression of an enzyme herein preferably means that activity of the enzyme is increased by at least 10%, 20%, 30%, 40%, 50%, 60%, 70%, 80%, 90%, or at least 100%. The activity may be increased by at least 1-fold, 2-fold, 3-fold, 4-fold, 5-fold, 6-fold, 7-fold, 8-fold, 9-fold, 10-fold, 1 log, 2 log, 3 log, or at least 4 log. Preferred glyoxylate reductase and isocitrate lyase are the ones described elsewhere herein.

In the recombinant host cell for the production of extracellular glycolate, the host cell may overexpress phosphoglycolate phosphatase in view of the parent cell. A preferred phosphoglycolate phosphatase is the one described elsewhere herein.

In the recombinant host cell for the production of extracellular glycolate, the host cell may comprises a ribulose bisphosphate carboxylase (Rubisco) that has decreased selectivity for CO₂ over 02 (given by the specificity constant S_{c/o}=(k^{c}_{cat}/K_{c})/(k^{o}_{cat}/Kₒ)), with similar or higher intrinsic turnover rate (k^{o/c}_{cat}) compared to the native Rubisco of the parent host cell. This specificity constant S_{c/o} is a measure of the relative capacities of the enzyme to catalyse carboxylation and oxygenation of ribulose 1,5-bisphosphate. It is calculated, based on the turnover numbers (maximum per active site catalytic rates in units of s⁻¹) for carboxylation (k^{c}_{cat}) and oxygenation (k^{o}_{cat}), as well as K_{C} and K_{O}, which indicate the Michaelis constants (half-saturation concentrations in μM) for carboxylation and oxygenation, respectively. Preferably, the Rubisco is one as listed in Table 1. More preferably, the Rubisco has a polypeptide sequence that has at least 70% sequence identity with SEQ ID NO: 16, 18, 20, 86 or 87, 91 or 93.

In one embodiment, the recombinant host cell for the production of extracellular glycolate comprises both a ribulose bisphosphate carboxylase (Rubisco) that has decreased selectivity for CO₂ over O₂ (as described above) and a Rubisco that does not have a decreased selectivity for CO₂ over O₂. Preferably, the Rubisco that does not have a decreased selectivity for CO₂ over O₂ is a Rubisco that is endogenous to the host cell. In one embodiment, the endogenous Rubisco is the endogenous Rubisco from Synechocystis and/or the Rubisco with decreased selectivity for CO₂ over 02 has a polypeptide sequence that has at least 70% sequence identity with SEQ ID NO: 16, 18, 20, 86 or 87, 91 or 93.

TABLE 1
Various Rubisco's

| Rubisco | | S_{c/o} | k_{c}^{cat} * | SEQ |
|---|---|---|---|---|
| (mutation) | Organism | ((k^{c}_{cat}/K_{c})/(k^{o}_{cat}/Kₒ)) | (s⁻¹) | ID NO: |
| Wildtype | Synechococcus PCC6103 | 56.1 ± 2.3 | 8.0 ± 0.7 | 85 + 89 |
| Rubisco | | | | |
| Mutated | Synechococcus PCC6103 | 51.3 ± 0.8 | 17.9 ± 0.6 | 86 + 89 |
| Rubisco | | | | |
| (RbcL_{F140I}) | | | | |
| Mutated | Synechococcus PCC6103 | 52.1 ± 1.5 | 8.8 ± 0.8 | 87 + 89 |
| Rubisco | | | | |
| (RbcL_{F345I}) | | | | |
| Mutated | Synechococcus PCC6103 | 59.3 ± 0.0 | 8.4 ± 0.3 | 88 + 89 |
| Rubisco | | | | |
| (RbcL_{F140I/F345I}) | | | | |
| Wildtype | Rhodospirillum rubrum | 9.0 ± 0.3 | 12.3 ± 0.3 | 16 |
| Rubisco | | | | |
| Mutated | Rhodospirillum rubrum | 5.5 ± 0.3 | 9.8 ± 0.4 | 18 |
| Rubisco | | | | |
| (RbcM_{H44N}) | | | | |
| Wildtype | Archaeolobus fulgidus | 4 | 23.1 | 20 |
| Rubisco | | | | |
| Wildtype | Rhodopseudomonas | 13 | 6.7 | 91 |
| Rubisco | capsulatus | | | |
| Wildtype | Rhodobacter | 9 | | 93 |
| Rubisco | sphaeroides | | | |

References: (Durão et al., 2015; Kreel, 2008; Mueller-Cajar et al., 2007)

In the recombinant host cell for the production of extracellular, the host cell may express a Rubisco with a specificity constant S_{c/o}<55. Preferably, S_{c/o}<54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or S_{c/o}<5.

In the recombinant host cell for the production of extracellular glycolate, the host cell may express a type II or type III Rubisco. There are four forms of Rubisco found in nature. Only forms I, II and III catalyse the carboxylation or oxygenation of ribulose bisphosphate. Form I is the most abundant form, found in eukaryotes and bacteria. It forms a hexadecamer consisting of eight large (L) and eight small (S) subunits. This form of Rubisco tends to have a high specificity for CO₂ (S_{C/O}˜40-170), but relatively poor catalytic rate (k_{cat}). Form II of Rubisco contains only dimers of L subunits, and in contrast to form I of Rubisco, form II tends to have a higher k_{cat} but a lower specificity for CO₂ (S_{C/O}˜10-20) (Mueller-Cajar et al., 2007). Form III is found primarily in archae and is also comprised of dimers of L subunits (Tabita et al., 2008).

In an embodiment, the recombinant host cell for the production of extracellular glycolate expresses a Rubisco of Rhodospirillum rubrum, optionally comprising a H44N mutation. Preferably, the Rubisco has at least 70% sequence identity with SEQ ID NO: 16, 18, 20, 86 or 87. More preferably, the Rubisco has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity with SEQ ID NO: 16, 18, 20, 86, 87, 91 or 93. Most preferably, the Rubisco has a polypeptide sequence as set forward in SEQ ID NO: 16, 18, 20, 86, 87, 91 or 93. The recombinant host cell for the production of extracellular glycolate, preferably is a photosynthetic cell, including algae and cyanobacteria. Preferred photosynthetic host cells include but are not limited to the following genera: Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Drapamaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, HiIlea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, lsthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, SpumeIla, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium.

More preferred host cells are a Synechocystis or a Synechococcus, or an Anabaena species. The recombinant host cell for the production of extracellular glycolate is preferably a host cell expressing a heterologous Phosphoribulokinase (PRK).

The recombinant host cell for the production of extracellular glycolate is preferably a host cell selected from the group consisting of a bacterial cell, and a fungal cell, preferably a yeast cell. When the host cell is a bacterial host cell, the host cell is preferably an Escherichia spp., Streptomyces spp., Zymonas spp., Acetobacter spp., Citrobacter spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. or Pantoea spp. A preferred Escherichia spp. is Escherichia coli When the host cell is a fungal host cell, the host cell is preferably a Saccharomyces spp., Schizosaccharomyces spp., Pichia spp., Kluyveromyces spp., Candida spp., Talaromyces spp., Brettanomyces spp., Pachysolen spp., Debaryomyces spp., Yarrowia spp. Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., or a Trichoderma spp. A preferred fungal cell is a Saccharomyces spp. cell.

The host cells defined herein can conveniently be used for the production of extracellular glycolate. Accordingly, the invention further provides for, a process for the production of extracellular glycolate comprising;
culturing a host cell as defined herein under conditions conducive to the production of glycolate and, optionally,
purifying the glycolate from the culture broth.

The person skilled in the art knows how to culture the host cells defined herein and knows how to purify glycolate from a culture broth. The culture broth can e.g. be separated from the host cells by centrifugation or membrane filtration and can subsequently purified by e.g. removal of excess water. Preferably, the yield of the process is at least 0.1 gram glycolate per litre culture broth, more preferably at least 0.2, 0.3, 0.4, 0.5, 0.6, 0.7, 0.8, 0.9, 1, 2, 3, 4, 5, 6, 7, 8, 9, or at least 10 gram glycolate per litre culture broth.

### Definitions

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWlN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall lengths, local alignments, such as those using the Smith Waterman algorithm, are preferred.

Alternatively, percentage similarity or identity may be determined by comparing against public databases, using algorithms such as FASTA, BLAST, etc. Thus, the nucleic acid and protein sequences of the invention can further be used as a “query sequence” to perform a comparison against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at www.ncbi.nlm.nih.gov/.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. Examples of classes of amino acid residues for conservative substitutions are known to the person skilled in the art.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. When used to indicate the relatedness of two nucleic acid sequences the term “homologous” means that one single-stranded nucleic acid sequence may hybridize to a complementary single-stranded nucleic acid sequence. The degree of hybridization may depend on a number of factors, including the amount of identity between the sequences and the hybridization conditions such as temperature and salt concentration as discussed later.

The term “heterologous”, when used with respect to a nucleic acid (DNA or RNA) or protein, refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. A heterologous nucleic acid or protein is not endogenous to the cell into which it is introduced, but has been obtained from another cell or synthetically or recombinantly produced. Generally, though not necessarily, such nucleic acids encode proteins that are not normally produced by the cell in which the DNA is transcribed or expressed. Similarly, exogenous RNA encodes for proteins not normally expressed in the cell in which the exogenous RNA is present. Heterologous nucleic acids and proteins may also be referred to as foreign nucleic acids or proteins. Any nucleic acid or protein that one of skill in the art would recognize as heterologous or foreign to the cell in which it is expressed is herein encompassed by the term heterologous nucleic acid or protein. The term heterologous also applies to non-natural combinations of nucleic acid or amino acid sequences, i.e. combinations where at least two of the combined sequences are foreign with respect to each other.

Any reference to nucleotide or amino acid sequences accessible in public sequence databases herein refers to the version of the sequence entry as available on the filing date of this document. In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a product or a composition may comprise additional component(s) than the ones specifically identified; said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

All patent and literature references cited in the specification are hereby incorporated by reference in their entirety.

The following examples are offered for illustrative purposes only, and are not intended to limit the scope of the invention in any way.

### EXAMPLES

### Example 1. Culture Conditions

Escherichia coli strains XL-1 blue (Stratagene), Turbo (NEB) or CopyCutter EP1400 (Epicentre biotechnologies) were used for plasmid amplification and manipulation, grown at 37° C. in Lysogeny Broth (LB) or on LB agar. Strains of Synechocystis sp. PCC 6803 and Synechococcus elongatus PCC 7942 were cultivated either on BG-11 plates or in BG-11 medium (Sigma Aldrich) optionally supplemented with 10 mM TES-KOH (pH=8), and/or 10 mM bicarbonate. BG-11 agar plates were supplemented with 10 mM TES-KOH (pH=8), 0.3% (w/v) sodium thiosulfate and 5 mM glucose. Strains of Synechococcus PCC 7002 were cultivated in the same medium as Synechocystis, but supplemented with 4 μg/l cyanocobalamin. When appropriate, the following antibiotics were used: ampicillin (100 μg/ml), kanamycin (20 or 50 μg/ml, for Synechocystis and E. coli, respectively), spectinomycin (25 μg/ml), streptomycin (10 μg/ml), and chloramphenicol (20 μg/ml). Strains were grown in Erlenmeyer flasks at 30° C., shaking 120 rpm. Alternatively, the strains were grown in the MC-1000 cultivator (Photon System) or in a 10 ml culture vial (CelIDEG), according to manufacturers' protocols. At several time points samples were taken from the culture vessel to analyze cell density and product formation by HPLC analysis, using a UV and RI detector.

### Example 2. General Protocols

Restriction endonucleases were purchased from Thermo Scientific. Amplification for high fidelity reactions used for cloning or sequencing was performed using Herculase II Fusion polymerase (Agilent), using a Biometra TRIO thermocycler. Primers used are mentioned in Table 2. Cloning was performed in E. coli using CaCl2)-competent XL1-blue, Turbo or CopyCutter EPI400 cells, according to manufacturer protocol.

Natural transformation for genomic integration of exogenous genes or deletion of endogenous genes in Synechocystis was performed using plates with increasing concentrations of antibiotic for growing the transformants to drive segregation. In the case of making markerless mutants, we used the mazF gene, encoding an endoribonuclease, driven by a Ni²⁺ inducible promoter system that allows for counter-selection (Cheah et al., 2013). This PBRS-mazF cassette [SEQ ID NO: 33] was synthesized at a gene synthesis company (GenScript) and then combined with different antibiotic markers. This cassette was used in combination with homologous regions targeting a specific part of the genome, first introducing and fully segregating it, before removing the marker based on Ni²⁺ selection.

Conjugation of RSF1010-based plasmids from E. coli XL-1 to Synechocystis was performed by tri-parental mating using E. coli J53 (pRP4) as the helper strain. Correct insertion of the genes and full segregation, as well as insertion of conjugation plasmids, were verified by colony PCR with specific primers (Table 2) and MyTaq DNA polymerase (Bioline).

### Example 3. Production of Glycolate Through Deletion of Glycolate Dehydrogenase

The genome of Synechocystis contains two glycolate dehydrogenase genes: s110404 (glcD1) [SEQ ID NO: 1, 2] and slr0806 (glcD2) [SEQ ID NO: 3, 4]. While we have deleted the whole glcD1 gene, we left some of the glcD2 intact as there is an antisense RNA present in the sequence, for which we wanted to preserve the function. To enable deletion, we have amplified the homologous regions (˜1000 bp) surrounding the genes with specific primers (#1-8; Table 2), fused them by fusion PCR while introducing restriction sites, and inserted this sequence into a pUC-18 backbone. Next, the Omega marker gene (conferring resistance to spectinomycin) and the PBRS-mazF cassette, that allows counter-selection to create markerless deletions, were inserted into these vectors.

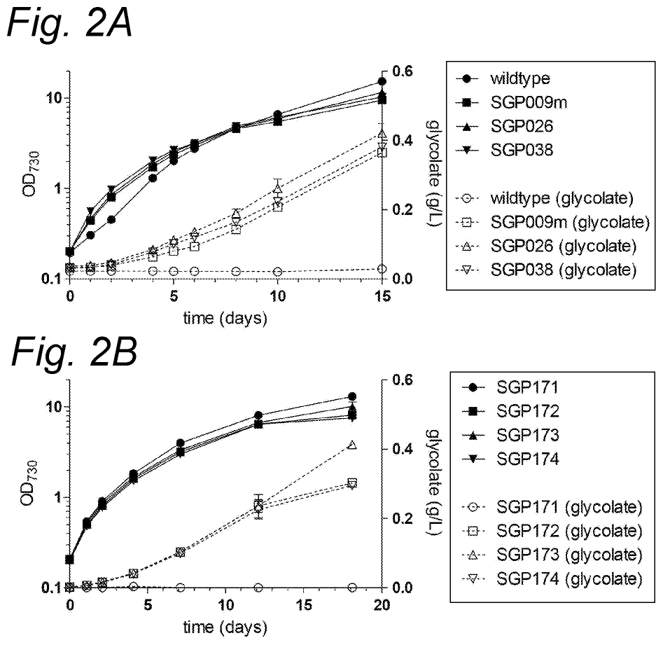

The resulting vectors were transformed into Synechocystis, first introducing and fully segregating the Δsll0404::spR. After making the resulting strain fully markerless (ΔglcD1), we introduced the next construct Δslr0806::spR and fully segregated the resulting strain. This strain was then again made fully markerless and was named SGP009m (ΔglcD1/2). After culturing the strain, we established that it was accumulating extracellular glycolate (FIG. 2a). The productivity of the intermediate strains is mentioned in Table 3.

### Example 4. Production of Glycolate Through Overexpression of Phosphoglycolate Phosphatase

Genes encoding phosphoglycolate phosphatase (PGP) [SEQ ID NO: 7, 8, 9, 10, 11, 12] were inserted into a vector targeting the slr0168 gene in the Synechocystis genome, pHKH-RFP [SEQ ID NO: 35]. These genes were either synthesized with codon-optimization (Genscript) or amplified from their host genome with specific primers (#13-14; Table 2). The genes were expressed with one of the following promoters: Ptrc, PcpcBA, PrbcL or PpsbA2 [SEQ ID NO: 37,38,39,40]. The resulting constructs were introduced into SGP009m, and tested for production of glycolate. An example of one of these strains, SGP026 is shown in FIG. 2a. Other examples are mentioned in Table 3.

### Example 5. Production of Glycolate Through Overexpression of Glycolate Permease

The nucleotide sequence encoding glycolate permease [SEQ ID NO: 13, 14] was synthesized with codon-optimization (Genscript) and inserted with a PpsbA2 promoter [SEQ ID NO: 39] into a vector targeting the slr0168 gene in the Synechocystis genome, pHKH-RFP [SEQ ID NO: 35]. The resulting construct was introduced into SGP009m, and tested for productivity of glycolate. A result of one of those strains is shown in FIG. 2a. The productivity is also mentioned in Table 3.

### Example 6. Production of Glycolate Through Overexpression of Glyoxylate Reductase

The nucleotide sequence encoding glyoxylate reductase [SEQ ID NO: 29,30] was synthesized with codon-optimization (Baseclear) and inserted with a Ptrc1 promoter [SEQ ID NO: 37] into the broad host range RSF1010-derivative plasmid pAVO+(van der Woude et al., 2016) [SEQ ID NO: 36]. The resulting construct, as well as an empty pAVO+ was introduced into Synechocystis wildtype and the ΔglcD1/2 strain SGP009m through conjugation. The resulting strains were tested for productivity of glycolate, as shown in FIG. 2b. Here, it is shown that glycolate productivity in a strain with overexpression of glyoxylate reductase is comparable, but not additional to SGP009m.

### Example 7. Production of Glycolate with a Host Unable to Form Carboxysomes

To remove the capacity of Synechocystis for carboxysome formation, we removed one of the genes encoding a central carboxysome component, ccmM. To delete the ccmM gene [SEQ ID NO: 34], we amplified the homologous regions (˜1000 bp) surrounding the gene with specific primers (#36-39; Table 2), fused them by fusion PCR while introducing restriction sites, and inserted this sequence into a pBSKII+ vector. Next, the marker gene (conferring resistance to spectinomycin) and the PBRS-mazF cassette (allowing counter-selection to create markerless deletions) were inserted in this vector. The resulting vector was introduced in the mutant Synechocystis strain SGP026 using the spectinomycin marker, and, after full segregation was achieved, the marker was removed through recombination based on Ni²⁺ selection. The resulting strain SGP105 was tested for glycolate productivity (Table 3).

### Example 8. Production of Glycolate with Alternative Rubisco

Additional to the deletion of the glycolate dehydrogenase, also the gene encoding lactate dehydrogenase (slr1556) [SEQ ID NO: 5, 6] was deleted. To this end, we have amplified the homologous regions (˜1000 bp) surrounding the genes with specific primers (#15-18; Table 2), fused them by fusion PCR while introducing restriction sites, and inserted this sequence into a pUC-18 backbone. Next, the Omega marker gene (conferring resistance to spectinomycin) and the PBRS-mazF cassette, that allows counter-selection to create markerless deletions, were inserted into the vector. The resulting vector was introduced into SGP026. After full segregation of the construct, the deletion was made markerless, resulting in strain SGP201 (Table 3).

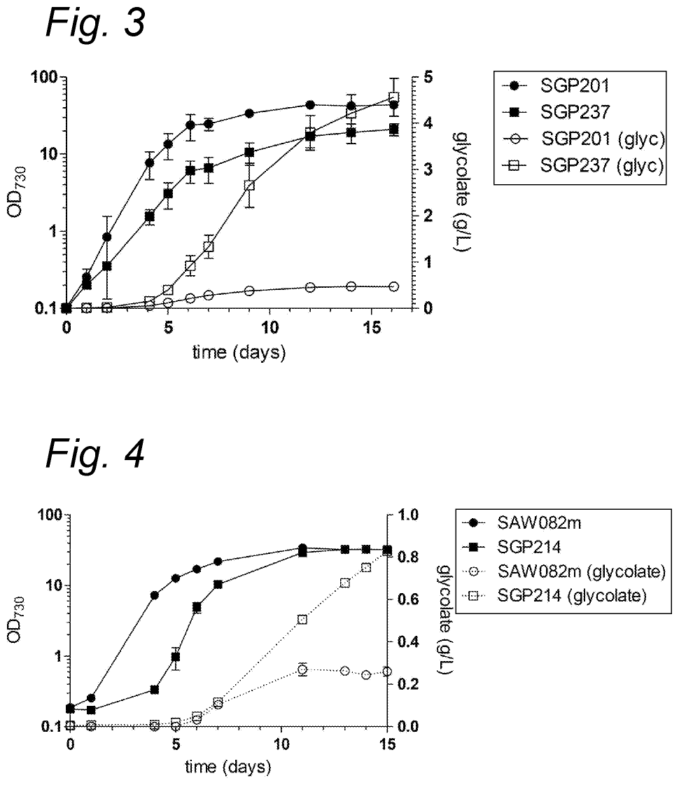

To replace the endogenous genes encoding the Rubisco operon [SEQ ID NO: 21] of Synechocystis, we amplified the homologous regions (˜1000 bp) surrounding the rbcLXS genes with specific primers (#25-28; Table 2), fused them by fusion PCR while introducing a number of restriction sites, and inserted this sequence in a pBSKII+ vector. Next, a nucleotide sequence encoding heterologous Rubisco, rbcM [SEQ ID NO: 15, 16], was amplified from Rhodospirillum rubrum with specific primers (#29-30; Table 2), and placed behind a PcpcBA promoter [SEQ ID NO: 38] inside the rbcLXS-targeting vector. Lastly, the marker gene (conferring resistance to chloramphenicol) and the PBRS-mazF cassette that allows counter-selection to create markerless deletions, were inserted in this vector. The resulting vector was used first to replace rbcLXS operon in the mutant Synechocystis strain SGP201 (Table 3) using the chloramphenicol marker, and, after full segregation was achieved, the marker was removed through recombination based on Ni²⁺ selection. The resulting strain was tested for glycolate productivity (FIG. 3).

### Example 9. Cyanobacterial Production of Glycolate Through the TCA Pathway

The nucleotide sequence encoding glyoxylate reductase [SEQ ID NO: 29,30] was synthesized with codon-optimization (Baseclear) and inserted in operon with a nucleotide sequence encoding isocitrate lyase[SEQ ID NO: 25,26], amplified with specific primers (#21-22; Table 2), driven by a Ptrc1 promoter [SEQ ID NO: 37] into the broad host range RSF1010-derivative plasmid pAVO+(van der Woude et al., 2016). The resulting construct was introduced into the ΔglcD1+ΔglcD2+Δldh strain SAW082m through conjugation, and tested for production of glycolate. A result of one of those strains is shown in FIG. 4. Productivity is also listed in Table 3.

### Example 10. Glycolate Production in Cell without PGPase

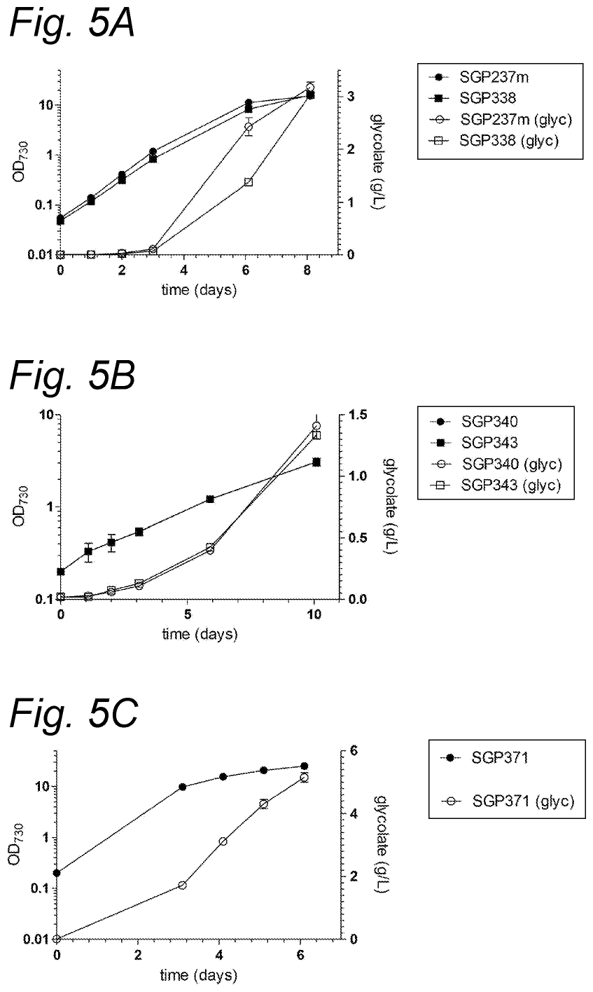

The PGPase overexpression cassette of SGP237 was replaced with only a kanamycin resistance marker, resulting in strain SGP338 (table 3). The strain was tested for productivity (FIG. 5A). This shows that PGPase is not essential for production of glycolate but the presence of Rubisco type II is sufficient.

### Example 11. Production of Glycolate with Type II Rubisco from Various Strains

To test multiple different Rubisco enzymes, nucleotide sequence encoding heterologous Rubisco, rbcM [SEQ ID NO: 90, 91, 92, 93], was amplified from Rhodopseudomonas capsulatus or Rhodobacter sphaeroides with specific primers (#29-30; Table 2), and placed behind a Pcpt promoter [SEQ ID NO: 94] inside the rbcLXS-targeting vector. These sequences were introduced at the same site as rbcMfrom R. rubrum (strain SGP237, table 3), the marker was removed through recombination based on Ni²⁺ selection. The resulting strains (SGP340 or SGP343) were tested for glycolate productivity (FIG. 5B).

### Example 12. Production of Glycolate in Synechocystis with Two Different Rubisco Enzymes

To make a strain with both form I and form II rubisco, we placed back the endogenous Rubisco in SGP237. To this end, the Rubisco operon [SEQ ID NO: 21] of Synechocystis was amplified with specific primers and cloned behind a behind a Ptrc promoter [SEQ ID NO: 37] in a vector targeting neutral site NSC2. The vector was introduced in SGP237 and the resulting strains (SGP340 or SGP343) were tested for glycolate productivity (FIG. 5C).

### Example 13. Production of Glycolate in Synechococcus PCC7002

To delete the gene encoding glycolate dehydrogenase in Synechococcus PCC7002 [SEQ ID NO: 95,96], we have amplified the homologous regions (˜1000 bp) surrounding the genes with specific primers (#15-18; Table 2), fused them by fusion PCR while introducing restriction sites, and inserted this sequence into a pBSKII+ vector. Next, the kanR marker gene (conferring resistance to kanamycin) and the PBRS-mazF cassette, that allows counter-selection to create markerless deletions, were inserted into the vector. The resulting vector was introduced into Synechococcus PCC7002 and full segregation resulted in strain ScGP001 (Table 4).

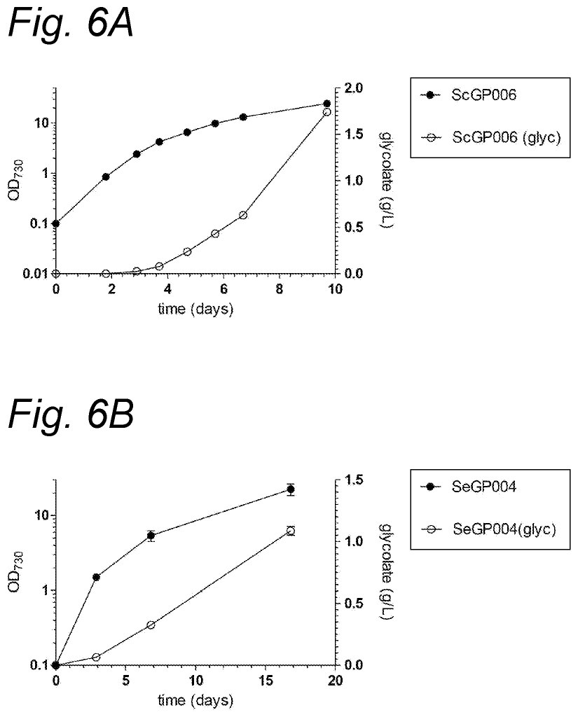

To replace the endogenous genes encoding the Rubisco operon [SEQ ID NO: 97] of Synechococcus PCC7002, we amplified the homologous regions (˜1000 bp) surrounding the rbcLXS genes with specific primers (#25-28; Table 2), fused them by fusion PCR while introducing a number of restriction sites, and inserted this sequence in a pBSKII+ vector. Next, a nucleotide sequence encoding heterologous Rubisco, rbcM [SEQ ID NO: 15, 16], was amplified from Rhodospirillum rubrum with specific primers (#29-30; Table 2) and placed behind a PcpcBA promoter [SEQ ID NO: 38] inside the rbcLXS-targeting vector. Lastly, the marker gene (conferring resistance to chloramphenicol) and the PBRS-mazFcassette that allows counter-selection to create markerless deletions, were inserted in this vector. The resulting vector was used first to replace rbcLXS operon in the mutant Synechococcus PCC7002 strain ScGP001 (Table 4) using the chloramphenicol marker and full segregation was achieved. The resulting strain ScGP006 was tested for glycolate productivity (FIG. 6A).

### Example 14. Production of Glycolate in Synechococcus elongatus PCC 7942

To replace the endogenous genes encoding the Rubisco operon [SEQ ID NO: 84] of Synechococcus elongatus PCC7942, we amplified the homologous regions (˜1000 bp) surrounding the rbcLXS genes with specific primers (#25-28; Table 2), fused them by fusion PCR while introducing a number of restriction sites, and inserted this sequence in a pBSKII+ vector. Next, a nucleotide sequence encoding heterologous Rubisco, rbcM [SEQ ID NO: 15, 16], was amplified from Rhodospirillum rubrum with specific primers (#29-30; Table 2) and placed behind a PcpcBA promoter [SEQ ID NO: 38] inside the rbcLXS-targeting vector, together with the marker gene camR (conferring resistance to chloramphenicol). The resulting vector was used to replace rbcLXSoperon in Synechococcus elongatus PCC7942 using the chloramphenicol marker and full segregation resulted in strain SeGP002 (Table 4).

To delete the gene encoding glycolate dehydrogenase in Synechococcus elongatus PCC7942 [SEQ ID NO: 101,102], we have amplified the homologous regions (˜1000 bp) surrounding the genes with specific primers (#15-18; Table 2), fused them by fusion PCR while introducing restriction sites, and inserted this sequence into a pBSKII+ vector. Next, we introduced a gene encoding phosphoglycolate phosphatase (PGP) [SEQ ID NO: 9, 10] behind a Ptrc promoter [SEQ ID NO: 37] and the kanR marker gene (conferring resistance to kanamycin) into the vector. The resulting vector was introduced into Synechococcus elongatus PCC7942 strain SeGP002 and full segregation was achieved. The resulting strain SeGP004 (Table 4) was tested for glycolate productivity (FIG. 6B).

TABLE 2
List of primers

| Nr | Primer | sequence |
|---|---|---|
| 1 | Hom1sll0404_F | cgtggtatctccatagctttg |
| 2 | Hom1sll0404_R | tcccttccccaccactagtccctaaaacaaaaaactgacaataatc |
| 3 | Hom2sll0404_F | ttttgttttagggactagtggtggggaagggaaaagtac |
| 4 | Hom2sll0404_R | gcttacaatcactcattggag |
| 5 | Hom1slr0806_F | gcatcaaaaatggtgcgtc |
| 6 | Hom1slr0806_R | tacttgccttggcactagtgctaagtctggattagtcg |
| 7 | Hom2slr0806_F | atccagacttagcactagtgccaaggcaagtaaagggg |
| 8 | Hom2slr0806_R | ccctctgtggccccgaag |
| 9 | slr0806_IN_F | ccacggctcaaaataacgtctttgc |
| 10 | slr0806_IN_R | ggcacatttgcccttgaatgcgc |
| 11 | sll0404_IN_F | cgtaggggcgtaggaggaacagg |
| 12 | sll0404_IN_R | gaaagcgccgatccatcctatggcc |
| 13 | Ndel_pgp_Syn_F | aaacatatgtggaaaagatcctggaaagc |
| 14 | BamHI_pgp_Syn_R | tttggatccctactgtcgcatcagttgcg |
| 15 | Slr1556-HOM1-F | cctgaatcgttatcggcact |
| 16 | Slr1556-HOM1-R | ggtttgcagagcgtttctagagctaaaatagcggtatcaag |
| 17 | Slr1556-HOM2-F | ataccgctattttagctctagaaacgctctgcaaaccattg |
| 18 | Slr1556-HOM2-R | cccaatccctaccggactat |
| 19 | slr1556_for | aaatttggggtgaagctggg |
| 20 | slr1556_rev | tgatgcgacaacaaaaggca |
| 21 | Nhel_RBS_Ndel_aceA | aaaagctagcattaaagaggagaaatgacatatgaaaacccgtacacaacaa |
| 22 | aceA_BamHI_AvrII | aaaacctaggggatccttattagaactgcgattcttcagtgg |
| 23 | Ndel-rbcL-7942F | aaaaaacatatgcccaagacgcaatctgc |
| 24 | BamHI-rbcS-7942R | aaaaaaggatccttagtagcggccgggacg |
| 25 | Rbc-HR1-F | ctggaaattctgtcagcggg |
| 26 | Rbc-HR1-R | gtaacgtcgacctgcagactagtgatatccatatgtctagactaggtcagtcctccataaac |
| 27 | Rbc-HR2-F | cctagtctagacatatggatatcactagtctgcaggtcgacgttacagttttggcaattactaaa |
| 28 | Rbc-HR2-R2 | aaccgtgccaattttcacct |
| 29 | RbcM_Rr_Ndel_F | aaaacatatggaccagtcatctcgttac |
| 30 | RbcM_Rr_Spel_R | aaaaactagtttacgccggaagggcgct |
| 31 | RbcM_H44N_F | gcggcgaatttcgccgccgagagttcg |
| 32 | RbcM_H44N_R | ggcgaaattcgccgcggtcgccac |
| 33 | RbcX-5UTR-Nhel-F | aaaagctagcattaacagcggcttaactaacag |
| 34 | Xbal-cpcBA-F | aaatctagacataaagtcaagtaggag |
| 35 | RbcX-BgIII-F | aaaaagatctatgcaaactaagcacatagct |
| 36 | Hom1_sll1031_F | agattttgccccatcaacag |
| 37 | Hom1_sll1031_R | gaacccgattctagataattactagttgaccagcccc |
| 38 | Hom2_sll1031_F | gtcaactagtaattatctagaatcgggttcaaatatg |
| 39 | Hom2_sll1031_R | agtccataccgtcgatgtcc |
| 40 | RbcL_F140l_F | tccgcatccccgtcgcc |
| 41 | RbcL_F140l_R | ggcgacggggatgcgga |
| 42 | RbcL_F345l_F | accttgggcattgttgacttg |
| 43 | RbcL_F345l_R | caagtcaacaatgcccaaggt |
| 44 | RbcRs_Ndel_F | atctcatatgatggaccagtccaaccgct |
| 45 | RbcRs_Spel_R | cgatactagttcaggccgcgcgatgcag |
| 46 | RbcRp_Ndel_F | atctcatatgatgcgatgcgcgacatctg |
| 47 | RbcRp_Spel_R | acatactagtttacgccgcctgcggctt |
| 48 | 7002glcD1-HOM1-f | tgtgctacttacccttgtcc |
| 49 | 7002glcD1- | tcatctcccagcacttttggtctagattttttactagttttcaggaaagcacagtggtttc |
| | HOM1overlap-R | |
| 50 | 7002glcD1- | gctttcctgaaaactagtaaaaaatctagaccaaaagtgctgggagatga |
| | HOM2overlap-F | |
| 51 | 7002glcD1-HOM2-R | ttacggttcgctcccattag |
| 52 | UTEXglcD1-HOM1-f | tttctcccgttgcattggcg |
| 53 | UTEXglcD1- | cgtgactgaaattccagctctctagattttttactagttttgacacagacgaactgttgcc |
| | HOM1overlap-R | |
| 54 | UTEXglcD1- | gtctgtgtcaaaactagtaaaaaatctagagagctggaatttcagtcacg |
| | HOM2overlap-F | |
| 55 | UTEXglcD1-HOM2-R | gtcaatcatctttgcggatc |
| 56 | Rbc7002_HR1_F | cgagatccatgccggcgc |
| 57 | Rbc7002_HR1_R | aaccctcgagctgcagactagtgatatccatatgtctagagcggttttcctccagcaaaa |
| 58 | Rbc7002_HR2_F | gctctagacatatggatatcactagtctgcagctcgagggttttgttggtttttgtgacc |
| 59 | Rbc7002_HR2_R | gcggctttctcacccatgg |
| 60 | Rbc7942_HR1_F | gacagctcgtcagtttgagc |
| 61 | Rbc7942_HR1_R | ggctctcgagctgcagactagtgatatccatatgtctagagtcgtctctccctagagata |
| 62 | Rbc7942_HR2_F | gactctagacatatggatatcactagtctgcagctcgagagcctgatttgtcttgatagc |
| 63 | Rbc7942_HR2_R | agatcagcgatcgctcgca |

TABLE 3
Synechocystis PCC6803 strain list with glycolate
production titres in the extracellular medium

| Synechocystis | | Glycolate |
|---|---|---|
| recombinant | | production |
| strain | Genotype | titres (g/L) |
| SGP002m | ΔglcD1 | 0.29 |
| SGP009m | ΔglcD1 + ΔglcD2 | 0.36 |
| SGP026 | ΔglcD1 + ΔglcD2 + slr0168::Ptrc_coPGPCr | 0.44 |
| SGP027 | ΔglcD1 + ΔglcD2 + slr0168::PcpcBA_PGPsyn7942 | 0.41 |
| SGP038 | ΔglcD1 + ΔglcD2 + slr0168::PspBA2_coGlcAEc | 0.38 |
| SGP105 | ΔglcD1 + ΔglcD2 + slr0168::Ptrc_coPGPCr + ΔccmM | 0.95 |
| SGP171 | pAVO+_empty | 0.0 |
| SGP172 | ΔglcD1 + ΔglcD2 + pAVO+_empty | 0.30 |
| SGP173 | pAVO+_ptrc1_GlyR1At | 0.40 |
| SGP174 | ΔglcD1 + ΔglcD2 + pAVO+_ptrc1_GlyR1At | 0.30 |
| SAW082m | ΔglcD1 + ΔglcD2 + Δldh | 0.28 |
| SGP201 | ΔglcD1 + ΔglcD2 + Δldh + slr0168::Ptrc_coPGPCr | 0.47 |
| SGP214 | ΔglcD1 + ΔglcD2 + pAVO+_ptrc1_AceAEc_GlyR1At | 0.83 |
| SGP237 | ΔglcD1 + ΔglcD2 + Δldh + slr0168::Ptrc_coPGPCr + | 4.56 |
| | rbcLXS::PcpcBA_rbcMRr | |
| SGP246 | ΔglcD1 + ΔglcD2 + Δldh + slr0168::Ptrc_coPGPCr + | TBA |
| | rbcLXS::PcpcBA_rbcLS₇₉₄₂X₆₈₀₃ | |
| SGP247 | ΔglcD1 + ΔglcD2 + Δldh + slr0168::Ptrc_coPGPCr + | TBA |
| | rbcLXS::PcpcBA_rbcL_{F140I}S₇₉₄₂X₆₈₀₃ | |
| SGP248 | ΔglcD1 + ΔglcD2 + Δldh + slr0168::Ptrc_coPGPCr + | TBA |
| | rbcLXS::PcpcBA_rbcL_{F345I}S₇₉₄₂X₆₈₀₃ | |
| SGP249 | ΔglcD1 + ΔglcD2 + Δldh + slr0168::Ptrc_coPGPCr + | TBA |
| | rbcLXS::PcpcBA_rbcL_{F140I/F345I}S₇₉₄₂X₆₈₀₃ | |
| SGP338 | ΔglcD1 + ΔglcD2 + Δldh + slr0168::kanR + | 3.1 |
| | ΔrbcLXS::PcpcBA_rbcMRr | |
| SGP340 | ΔglcD1 + ΔglcD2 + Δldh + slr0168::Ptrc_coPGPCr + | 1.5 |
| | ΔrbcLXS::Pcpt_rbcMRs | |
| SGP343 | ΔglcD1 + ΔglcD2 + Δldh + slr0168::Ptrc_coPGPCr + | 1.4 |
| | ΔrbcLXS::Pcpt_rbcMRc | |
| SGP371 | ΔglcD1 + ΔglcD2 + Δldh + slr0168::Ptrc1_pgpCr + | 5.0 |
| | ΔrcbLXS::PcpcBA_rbcMRr + NSC2::Ptrc_rbcLXS | |

TABLE 4
Synechococcus strain list with glycolate
production titres in the extracellular medium

| Synechococcus | | | Glycolate |
|---|---|---|---|
| recombinant | | | production |
| strain | Background strain | Genotype | titres (g/L) |
| ScGP001 | Synechococcus PCC7002 | ΔglcD1::kanR | NT |
| ScGP006 | Synechococcus PCC7002 | ΔglcD1::kanR + | 1.76 |
| | | rbcLXS::PcpcBA_rbcMRr | |
| SeGP002 | Synechococcus | ΔrbcLXS::PcpcBA_rbcMRr | NT |
| | elongatus PCC7942 | | |
| SeGP004 | Synechococcus | ΔglcD1::Ptrc_coPGPCr + | 1.1 |
| | elongatus PCC7942 | ΔrbcLXS::PcpcBA_rbcMRr | |

NT: not tested

### REFERENCES

Cheah, Y. E., Albers, S. C., and Peebles, C. A. M. (2013). A novel counter-selection method for markerless genetic modification in Synechocystis sp. PCC 6803. Biotechnol. Prog. 29, 23-30.
Durão, P., Aigner, H., Nagy, P., Mueller-Cajar, O., Hartl, F. U., and Hayer-Hartl, M. (2015). Opposing effects of folding and assembly chaperones on evolvability of Rubisco. Nat. Chem. Biol. 11, 148-155.
He, Y.-C., Xu, J.-H., Su, J.-H., and Zhou, L. (2010). Bioproduction of Glycolic Acid from Glycolonitrile with a New Bacterial Isolate of Alcaligenes sp. ECU0401. Appl. Biochem. Biotechnol. 160, 1428-1440.
Kreel, N. E. (2008). Examination of Mutants that Alter Oxygen Sensitivity and CO_{2/02} Substrate Specificity of the Ribulose 1,5-Bisphosphate Carboxylase/Oxygenase (Rubisco) from Archaeoglobus fulgidus. The Ohio State University.
Loder, J. D. (1939). Process for manufacture of glycolic acid.
Mueller-Cajar, O., Morell, M., and Whitney, S. M. (2007). Directed evolution of rubisco in Escherichia coli reveals a specificity-determining hydrogen bond in the form II enzyme. Biochemistry 46, 14067-14074.
Tabita, F. R., Satagopan, S., Hanson, T. E., Kreel, N. E., and Scott, S. S. (2008). Distinct form I, II, III, and IV Rubisco proteins from the three kingdoms of life provide clues about Rubisco evolution and structure/function relationships. J. Exp. Bot. 59, 1515-1524.
Taubert et al. (2019) Glycolate from microalgae: an efficient carbon source for biotechnological applications. Plant Biotechnology Journal. doi: 10.1111/pbi.13078.
Wei, G., Yang, X., Gan, T., Zhou, W., Lin, J., and Wei, D. (2009). High cell density fermentation of Gluconobacter oxydans DSM 2003 for glycolic acid production. J. Ind. Microbiol. Biotechnol. 36, 1029-1034.
van der Woude, A. D., Perez Gallego, R., Vreugdenhil, A., Puthan Veetil, V., Chroumpi, T., and Hellingwerf, K. J. (2016). Genetic engineering of Synechocystis PCC6803 for the photoautotrophic production of the sweetener erythritol. Microb. Cell Factories 15, 60.

1. A recombinant host cell for the production of extracellular glycolate, wherein the host cell:
is derived from a parent host cell,
comprises phosphoribulokinase (PRK) and ribulose bisphosphate carboxylase (Rubisco) activity,
is substantially unable to anabolize glycolate, optionally,
comprises increased phosphoglycolate phosphatase activity compared to the parent host cell, and optionally,
comprises a permease to export glycolate out of the host cell into the culture medium.

2. A recombinant host cell or the production of extracellular glycolate according to claim 1, wherein the ribulose bisphosphate carboxylase (Rubisco) activity is with increased sensitivity for O2 compared to the Rubisco of the parent cell.

3. The recombinant host cell for the production of extracellular glycolate according to claim 1, wherein the host cell is substantially unable to metabolize glycolate due to reduced or eliminated glycolate dehydrogenase, glycolate oxidase activity and/or lactate dehydrogenase activity in view of the parent cell.

4. The recombinant host cell for the production of extracellular glycolate according to claim 1, wherein the reduced or eliminated glycolate dehydrogenase and/or glycolate oxidase activity in view of the parent cell is due to targeted gene disruption of deletion of a glycolate dehydrogenase and/or glycolate oxidase and/or lactate dehydrogenase.

5. The recombinant host cell for the production of extracellular glycolate according to claim 1, wherein the host cell overexpresses glyoxylate reductase and/or isocitrate lyase in view of the parent cell.

6. The recombinant host cell for the production of extracellular glycolate according to claim 1, wherein the host cell overexpresses phosphoglycolate phosphatase in view of the parent cell.

7. The recombinant host cell for the production of extracellular glycolate according to claim 1, wherein the host cell comprises a ribulose bisphosphate carboxylase (Rubisco) that lias decreased selectivity for CO2 over O2 (given by the specificity constant Sc/o=(kccat/Kc)/(kocat/Ko)), with similar or higher intrinsic carboxylation rate (kccat) compared to the native Rubisco of the parent host cell.

8. The recombinant host cell for the production of extracellular glycolate according to claim 1, wherein the host cell expresses a Rubisco with a specificity constant Sc/o<55.

9. The recombinant host cell for the production of extracellular glycolate according to claim 1, wherein the host cell expresses a type II or type III Rubisco

10. The recombinant host cell for the production of extracellular glycolate according to claim 1, wherein the host cell expresses a Rubisco of Rhodospirillum rubrum, optionally comprising a H44N mutation.

11. The recombinant host cell for the production of extracellular glycolate according to claim 1, wherein the Rubisco has at least 80% sequence identity with SEQ ID NO: 16, 18, 20, 86, 87, 91 or 93.

12. The recombinant host cell for the production of extracellular glycolate according to claim 1, wherein the host cell is a photosynthetic cell, preferably a cyanobacterium, preferably a Synechocystis or a Synechococcus, or an Anabaena species.

13. The recombinant host cell for the production of extracellular glycolate according to claim 1, wherein the host cell is a host cell expressing a heterologous Phosphoribulokinase (PRK).

14. The recombinant host cell for the production of extracellular glycolate according to claim 13, wherein the host cell is a host cell selected from the group consisting of a bacterial cell and a fungal cell.

15. A process for the production of extracellular glycolate comprising,
culturing a host cell according to claim 1 under conditions conducive to the production of glycolate and, optionally,
purifying the glycolate from the culture broth.

16. The process according to claim 15, wherein the yield of extracellular glycolate is higher than 1 gram glycolate per litre culture broth.

17. The recombinant host cell according to claim 7, wherein the host cell comprises a Rubisco that has a polypeptide sequence that has at least 70% sequence identity with a Rubisco listed in Table 1.

18. The recombinant host cell according to claim 7, wherein the host cell comprises a Rubisco that has a polypeptide sequence that has at least 70% sequence identity with SEQ ID NO: 16, 18, 20, 86, 87, 91 or 93.

19. The recombinant host cells according to claim 14, wherein the host cell is an Escherichia coli cell.

20. The recombinant host cells according to claim 14, wherein the host cell is a yeast cell, preferably a Saccharomyces spp. cell.


SEQUENCE LISTING (122 sequences)

SEQ ID NO: 1 - DNA, 1479 bp, Synechocystis PCC6803
atggccattttctcccccgtcaacgccgttaccgatattattccccagctcgaaaaaattgttggccaggatggagtaattaaacgcaaagacgagctattcacctacgaatgcgacggtttaacgggttatcgacaacggccggccctggtggttttgccccgcacaacggaacaggtagccacaatagtgaaactttgtcacgatcgccaaattccttggattgccaggggggctggcacagggttatcggggggagccttgccgggggccgatagcctattgattgtcaccactcgcatgcggcaaattttggcagtagattacgacaaccagaccattgttgtccagccgggggtggtgaataactgggttacccaaaccgttagtggggctggcttttactatgcccctgatccttccagtcagattgtctgctccattggcggtaatattgcggaaaattccggtggagttcattgtttgaaatatggcaccaccaccaaccatgtgctgggcttgaaactggttattcccgatggctccattgtggaagtaggggggcaagtccccgaaacgccgggctacgatttaaccggtttatttgttggttccgaaggaaccctaggcatcgccacagaaatcaccctaaaaattctcaaaaccccagaatctatctgtgtcgtattggcggattttctttctctcgaagccaccgcccaatccgtggccgatatcattgcggcgggcatcgtcccagcgggcatggaaattatggacaatttcagcatcaatgcggtggaagacgtggtggccaccaattgttaccccagggatgcggcggccattttgttagtggaactggacggtctgcccatcgaagtggaattaaaccaagccaaagtagaagaaatttgccgcaacaatggagcccgcaacacggcgatcgcctacgaccaagaaacccgcctaaaaatgtggaaaggaagaaaagcggcctttgcggcggcgggtaaactaagccccagttactttgtccaagatggtgtggtaccccggactcaattggtacagattttaagcgacattaatgatttaagtaagaaatatggctttgccattgccaatgttttccatgccggagacggtaatttacatcccctaattttgtatgatcaaaaagtaccaggagcctgggaaaaagtggaagaattggggggagaaatccttaaacgctgtgtggaattggggggaagtttatccggagaacacggcattggcattgataaaaattgctttatgcccaatatgttcaacgaagtagatttagaaacaatgcaatgggtcagacaatgttttaatcctgataacttagctaatcctggtaagctttttcctaccccccgcagttgtggagaagtggccaatgcccaacggcttaacctaggccaggacaagaaaatggaggaaatttattga

SEQ ID NO: 2 - protein, 492 aa, Synechocystis PCC6803
MAIFSPVNAVTDIIPQLEKIVGQDGVIKRKDELFTYECDGLTGYRQRPALVVLPRTTEQVATIVKLCHDRQIPWIARGAGTGLSGGALPGADSLLIVTTRMRQILAVDYDNQTIVVQPGVVNNWVTQTVSGAGFYYAPDPSSQIVCSIGGNIAENSGGVHCLKYGTTTNHVLGLKLVIPDGSIVEVGGQVPETPGYDLTGLFVGSEGTLGIATEITLKILKTPESICVVLADFLSLEATAQSVADIIAAGIVPAGMEIMDNFSINAVEDVVATNCYPRDAAAILLVELDGLPIEVELNQAKVEEICRNNGARNTAIAYDQETRLKMWKGRKAAFAAAGKLSPSYFVQDGVVPRTQLVQILSDINDLSKKYGFAIANVFHAGDGNLHPLILYDQKVPGAWEKVEELGGEILKRCVELGGSLSGEHGIGIDKNCFMPNMFNEVDLETMQWVRQCFNPDNLANPGKLFPTPRSCGEVANAQRLNLGQDKKMEEIY

SEQ ID NO: 3 - DNA, 1320 bp, Synechocystis PCC6803
atggattggtcagccattgcggcttcattaactacccagggcttggaagtaatccaagatccccagcaaagaaaaaagctatccaccgattacgcccatttcagccccattttgatggcgcagttggagggcaaacaggcggatttagtcgtcctagcccgatcagaaccagaggcaatcgcggtgatccgttgctgtgtggccaatcaaattcccctcacagtgcggggggctggcaccggcaattacggccaatgtgttcccctggagggaggcattgtgttggatttatcccccatgcaaaggatcattagcctggaaccgggtcgggccgtggtggagccgggggtaaagttaggcaagctggaacaacaagctaaacaaatgggctgggagctgcgcctgttaccttccacctatcaaacagctaccgtggggggttttgtcagcggtggcagtactggcatgggggcagtgaactatggcaccttattcgacccgggcaacgtccagagcctcacggtgttgaccatggaagcggaaccccaacggctgattctgtccggcgaggcggcccaaccagtcatccatggctatggcaccaacggcattattactgaaattactttgccccttactccagccctaccttggcgggaagccatcgtcagttttacgaatttatcttcggcgatcgcctttgcccaaaatttggcccatcaagacggcattgttagtaaggaaatttccattcaagccgatccgattccgcaatatttcagcagtttaaaaagttattatcaacccggagctcactgggtaatggtgattgtttctgagttggattggctagcttttacgcagttggccaaggcaagtaaaggggaaattatctttgagcaagatccccaaagcccagggaaaaaaattaatttgattgaatttaattggaaccacaccactcttttggctagggccgtggatcctagtttgacctatcttcaagtatttttctatcgagatgtagagcaaattctcgccttggcaaaactatttaaggatgagattatgttccacatcgaaataatgcgcattcaagggcaaatgtgcctagctggttttcctttggttaaatttatcaacggtgatcgcctggaagaaatcatggccgcccaccaaaatctaggggccagaatcgccaatccccacacctatagtttagccggagggtcggtacaacctctgccagaatcccaattaatttttaaacgccaggtagatccccttaatttgctcaacccaggtaaattaaccgattaa

SEQ ID NO: 4 - protein, 439 aa, Synechocystis PCC6803
MDWSAIAASLTTQGLEVIQDPQQRKKLSTDYAHFSPILMAQLEGKQADLVVLARSEPEAIAVIRCCVANQIPLTVRGAGTGNYGQCVPLEGGIVLDLSPMQRIISLEPGRAVVEPGVKLGKLEQQAKQMGWELRLLPSTYQTATVGGFVSGGSTGMGAVNYGTLFDPGNVQSLTVLTMEAEPQRLILSGEAAQPVIHGYGTNGIITEITLPLTPALPWREAIVSFTNLSSAIAFAQNLAHQDGIVSKEISIQADPIPQYFSSLKSYYQPGAHWVMVIVSELDWLAFTQLAKASKGEIIFEQDPQSPGKKINLIEFNWNHTTLLARAVDPSLTYLQVFFYRDVEQILALAKLFKDEIMFHIEIMRIQGQMCLAGFPLVKFINGDRLEEIMAAHQNLGARIANPHTYSLAGGSVQPLPESQLIFKRQVDPLNLLNPGKLTD

SEQ ID NO: 5 - DNA, 1002 bp, Synechocystis PCC6803
atgaaaatcgctttttttagcagtaaagcctatgatcgtcaatttttccaacaagcaaaccacccccatcaacgggaaatggtcttttttgatgcccaactcaaccttgataccgctattttagcggaggattgccccgttatttgcctcttcgttaatgaccaagctcctgccccggtgctagaaaagttagctgcccagggcacaaaattaatcgctctgcgcagtgcgggctataataatgttgacctcaaaacagccgcagatctggggctaaaagtagtccatgtaccatcctattctccccatgcagtcgcagaacatactgtggggttaattctggctctaaatagaaagttatatcgagcctataaccgcgttagggacgataatttttccctagaagggttacttggttttgatctccatggcaccaccgtcggagtgattggtactggcaaaattggtctagcttttgctcaaattatgaatggttttggctgccatttattgggctacgatgcctttcctaacgataaatttactgccattggccaagccctctatgtatctttaaatgaacttttagctcattctgatattatttctctccactgtcccctactgccggaaacccattatctaattaacactaataccattgcccaaatgaagccaggggttatgttaatcaacacaagtcgaggtcatttaattgatacccaagcggttattcaggggattaaatcccataaaattggctttttaggcattgatgtttacgaagaagaggaagagctattttttactgaccattctgatactatcattcaagatgatacctttcaattgctacaatcttttcccaatgtaatgatcacagctcatcagggattctttacccacaacgctctgcaaaccattgccgcaacgacactggcaaatattgctgaatttgagcagaataaacctttaacttaccaagtaatctgtccccattaa

SEQ ID NO: 6 - protein, 333 aa, Synechocystis PCC6803
MKIAFFSSKAYDRQFFQQANHPHQREMVFFDAQLNLDTAILAEDCPVICLFVNDQAPAPVLEKLAAQGTKLIALRSAGYNNVDLKTAADLGLKVVHVPSYSPHAVAEHTVGLILALNRKLYRAYNRVRDDNFSLEGLLGFDLHGTTVGVIGTGKIGLAFAQIMNGFGCHLLGYDAFPNDKFTAIGQALYVSLNELLAHSDIISLHCPLLPETHYLINTNTIAQMKPGVMLINTSRGHLIDTQAVIQGIKSHKIGFLGIDVYEEEEELFFTDHSDTIIQDDTFQLLQSFPNVMITAHQGFFTHNALQTIAATTLANIAEFEQNKPLTYQVICPH

SEQ ID NO: 7 - DNA, 759 bp, Escherichia coli
atgaataagtttgaagatattcgcggcgtcgcttttgatctcgatggaaccctggttgactccgcgcccggtctggctgccgcagtagatatggccctgtatgctcttgaactaccggtagcgggcgaggaacgcgttattacgtggattggaaatggggctgacgtattgatggaacgcgctttaacttgggcccggcaagaaagagccacccagcgaaagacaatgggcaaaccgcccgtggacgatgacattcctgccgaagagcaggtccggattctgcgtaaactatttgatcgctactatggtgaagtcgcggaagaggggaccttcttatttccccatgtggctgataccttgggagccttgcaggccaaagggctccccttgggtttagttaccaataagcccacccccttcgttgcgcccctgctcgaagccctcgatattgccaaatactttagcgtggtcattggtggcgatgacgtgcaaaacaagaaaccccaccccgatcctttgctgttagtcgcggaacggatgggtatcgccccacagcaaatgcttttcgtgggtgattctcgtaacgacatccaggctgcaaaagctgccggttgtcctagcgtgggtctaacttacggctataattacggggaagccattgatttgagtcaaccagatgtgatctatcaaagtatcaacgacttgctcccggccttaggcttgccccactccgaaaaccaagaatccaaaaacgattaa

SEQ ID NO: 8 - protein, 252 aa, Escherichia coli
MNKFEDIRGVAFDLDGTLVDSAPGLAAAVDMALYALELPVAGEERVITWIGNGADVLMERALTWARQERATQRKTMGKPPVDDDIPAEEQVRILRKLFDRYYGEVAEEGTFLFPHVADTLGALQAKGLPLGLVTNKPTPFVAPLLEALDIAKYFSVVIGGDDVQNKKPHPDPLLLVAERMGIAPQQMLFVGDSRNDIQAAKAAGCPSVGLTYGYNYGEAIDLSQPDVIYQSINDLLPALGLPHSENQESKND

SEQ ID NO: 9 - DNA, 993 bp, Chlamydomonas reinhardtii
atgctatccctcaaacaattacccagcgcgcggtgtgctgcccgtcctgtacggcccgtgcggcgcatggttgccgcgcaagcttccgctcggcccattgccaccaacgaacagaaactggaattattgaagaaagtggaatgctttatcttcgattgcgatggtgttatttggctcggtgacaaagtgattgaaggtgtgcctgaaaccttggatatgctgcgtggtatgggtaagaaagtattcttcgtgactaataactctaccaagtcccgcgctggttacatgtccaaatttcagagcttgggcttgaatgtgaaggcggaagaaatctactccagctcttacgccgctgccgcttatttggaatccatcaactttaacaagaaggtttatgtgatcggtgaaactggcattctggaagaattagacctgaaaggtatccgtcacgttggtggtcccggcgacgctgataagaaggttacgttgaaatccggtgaatttatggaacacgaccatgacgtgggtgccgtcgtagtcgggtttgatcgctacgtcaattattacaagatccaatatgctaccttgtgtattcgtgagaatccgggctgtatgttcattgcgaccaatcgtgacgccgtcacccacttgaccgacgcccaggaatgggccggtaacggcagtatggtgggtgccattgtcggttccacgaagcgtgaacccatcgtagtgggcaaacccagtgactttatgctcaagaatatttccgccagtttagggttgcgccctgaccaaattgctatggtaggtgatcgtttggataccgatattatgtttggcaagaacggcggtctagcgaccgccctcgtcttgtctggtgtgaccactcccgaagttctgaatagcccggataacaaagtgcatcccgatttcgtattaaactccttacccgatttgctgagtgttaaagagaaggctatggtggccgcctaa

SEQ ID NO: 10 - protein, 330 aa, Chlamydomonas reinhardtii
MLSLKQLPSARCAARPVRPVRRMVAAQASARPIATNEQKLELLKKVECFIFDCDGVIWLGDKVIEGVPETLDMLRGMGKKVFFVTNNSTKSRAGYMSKFQSLGLNVKAEEIYSSSYAAAAYLESINFNKKVYVIGETGILEELDLKGIRHVGGPGDADKKVTLKSGEFMEHDHDVGAVVVGFDRYVNYYKIQYATLCIRENPGCMFIATNRDAVTHLTDAQEWAGNGSMVGAIVGSTKREPIVVGKPSDFMLKNISASLGLRPDQIAMVGDRLDTDIMFGKNGGLATALVLSGVTTPEVLNSPDNKVHPDFVLNSLPDLLSVKEKAMVAA

SEQ ID NO: 11 - DNA, 822 bp, Synechococcus elongatus
atgtggaaaagatcctggaaagctttcatagcaagactttcagcgatcgacccctgtggaaaagctgtggaaaaactaagagggttttccacaggggggatcaaggggggttcctttctccacagactgaaggggttttccacaagaaatccacaggtttttccacagagttatccacagactttgcaggcgattatttttgattttgatggaactttagtagattctctgcctactgtagttgcaatcgctaatgctcatgccccggattttggttatgacccgatcgatgagcgtgactatgcgcaactgcgtcagtggtcttcccgcacgatcgtgcggcgtgcgggtctgtcaccttggcagcaggcgcggttactccaacgggtgcaacgccagctaggggattgtctaccggcgctgcagctctttcctggggttgcagacctcttggctcaactgcgatcgcgatcgctctgtcttgggattcttagctccaacagtcggcagaacatcgaagcctttttgcaacgacaaggtctgcgatcgctgttctctgtcgttcaagctggaacgcccattttgagtaagcgtcgggctctcagtcagttggtggctcgcgagggctggcagccagcagctgtgatgtatgtcggcgatgaaacccgcgatgtggaagctgctcgtcaggtgggtctgattgctgtggccgtgacttggggctttaacgatcgccaaagcctggtcgcggcctgtcctgattggctactagaaactccctcagacctattgcaagctgtgacgcaactgatgcgacagtag

SEQ ID NO: 12 - protein, 273 aa, Synechococcus elongatus
MWKRSWKAFIARLSAIDPCGKAVEKLRGFSTGGIKGGSFLHRLKGFSTRNPQVFPQSYPQTLQAIIFDFDGTLVDSLPTVVAIANAHAPDFGYDPIDERDYAQLRQWSSRTIVRRAGLSPWQQARLLQRVQRQLGDCLPALQLFPGVADLLAQLRSRSLCLGILSSNSRQNIEAFLQRQGLRSLFSVVQAGTPILSKRRALSQLVAREGWQPAAVMYVGDETRDVEAARQVGLIAVAVTWGFNDRQSLVAACPDWLLETPSDLLQAVTQLMRQ

SEQ ID NO: 13 - DNA, 1683 bp, Escherichia coli
atggtgacctggacccagatgtacatgcctatgggtgggcttggattgagcgctttggtagccttgattcccatcatattcttctttgtggccttagctgtcttgcgtttgaaagggcacgtagccggtgcgatcacccttatcctgtctatcctgattgcgatatttgcctttaaaatgcccatcgacatggctttcgcggcagctgggtatggctttatctacggcctgtggcccatcgcgtggattatcgttgctgccgttttcctctacaaattgactgttgcctccggtcaatttgacatcattcgctccagtgttattagtatcaccgatgaccaacggttacaggtgctgttgattggtttctctttcggggctctgttggagggcgccgctggctttggagcacccgttgctattaccggagccttgctcgtgggcttggggttcaagcccttgtatgctgccggtctatgtttgattgcgaacaccgcgcccgtggccttcggggccctcggggtccccattctagtcgccggacaagtgaccggcattgatccctttcatatcggagcaatggctggacgacaattaccgttcttgagtgttcttgtgccgttttggttggtagctatgatggatggttggaaaggtgtgaaagaaacctggcctgccgcgctagtagccggtgggtccttcgcagttactcaattctttacctccaactacattggccccgaactccctgacatcacgtctgctctggtatcaattgtgagcttagcactgtttctaaaggtgtggcgcccgaagaacacggaaacggccattagcatggggcagtccgccggcgcaatggtcgttaacaaaccctccagcggcgggcccgtgccgtctgaatatagtctcggtcaaattatcagagcgtggagtccgttcctgattcttacagtattggttacgatttggactatgaagccttttaaggctttatttgctccaggcggcgcattttacagcctcgtgatcaattttcaaattccccacttgcaccagcaagtattgaaggctgcacccattgttgcccaacccaccccaatggatgcagtcttcaaattcgatcccctgtccgccggcggtacggccatatttatcgccgctattatttccatctttattcttggtgttggtattaagaagggtattggtgtgttcgcggagaccttaattagtctcaaatggccaatcctatcgattggcatggttttagcctttgcctttgtgaccaattactcggggatgagtaccactttggcgttggtcttagccggcactggcgtcatgttcccattcttctcaccattcctcggttggttaggcgtctttctaactggttccgacacttctagtaacgcattatttggaagcctacagtccaccacagcgcagcaaattaatgtatccgataccctgttagtggcagctaataccagcggtggcgtcaccggtaaaatgattagtcctcagtccatcgccgtggcctgcgccgccaccggtatggtgggtcgggaatccgaactcttccgttataccgtgaaacattccctgatctttgcctccgtgatcggtattatcactttactacaggcctatgtgttcaccggcatgctcgttagctaa

SEQ ID NO: 14 - protein, 560 aa, Escherichia coli
MVTWTQMYMPMGGLGLSALVALIPIIFFFVALAVLRLKGHVAGAITLILSILIAIFAFKMPIDMAFAAAGYGFIYGLWPIAWIIVAAVFLYKLTVASGQFDIIRSSVISITDDQRLQVLLIGFSFGALLEGAAGFGAPVAITGALLVGLGFKPLYAAGLCLIANTAPVAFGALGVPILVAGQVTGIDPFHIGAMAGRQLPFLSVLVPFWLVAMMDGWKGVKETWPAALVAGGSFAVTQFFTSNYIGPELPDITSALVSIVSLALFLKVWRPKNTETAISMGQSAGAMVVNKPSSGGPVPSEYSLGQIIRAWSPFLILTVLVTIWTMKPFKALFAPGGAFYSLVINFQIPHLHQQVLKAAPIVAQPTPMDAVFKFDPLSAGGTAIFIAAIISIFILGVGIKKGIGVFAETLISLKWPILSIGMVLAFAFVTNYSGMSTTLALVLAGTGVMFPFFSPFLGWLGVFLTGSDTSSNALFGSLQSTTAQQINVSDTLLVAANTSGGVTGKMISPQSIAVACAATGMVGRESELFRYTVKHSLIFASVIGIITLLQAYVFTGMLVS

SEQ ID NO: 15 - DNA, 1401 bp, Rhodospirillum rubrum
atggaccagtcatctcgttacgtcaatctggcgctcaaggaagaggatctgatcgctggcggcgagcatgtgctctgtgcctatatcctgaagcccaagcccggatatggctatgtggcgacagcggcgcatttcgccgccgaaagctcgacgggcaccaacgtcgaggtttgcaccaccgatgatttcacccggggcgtcgacgccctggtctatgaggtggacgaggcccgcgagctgaccaagatcgcctatccggtggctttgttcgaccgcaacatcaccgacggcaaggcgatgatcgcctcgttcctgacgctcaccatgggaaataaccagggtatgggcgacgtggaatacgccaagatgcacgatttctatgtgcccgactcctatcgcgccctgttcgatggccccagcgtcaatatctcggccctgtggaaggtgctaggtcggcccgaggtcgacggcggtttggtcgtcggcacgatcatcaagccgaagctcggcctgcgtcccaagcccttcgccgaggcctgccacgccttctggctgggcggcgatttcatcaagaacgacgagccccagggcaatcagcccttcgcccccttgcgcgacaccatcgccctggtcgccgacgccatgaagcgggcccaggacgagaccggcgaggccaagctgttctcggccaatatcaccgccgacgatcccttcgagatcatcgcgcgtggcgagtatgtgctgaagaccttcggcgagaacgcctcgcatgtcgccttgctggtcgacggctatgtcgccggcgccgcggcgatcaccacggcgcgccgccgtttccccgataacttcctgcattatcaccgggccggccacggcgccgtcacctcgccccagtccaagcgcggctataccgccttcgtccattgcaagatggcccgcctgcagggcgccagcggcatccaaaccggcaccatgggctttggcaagatggaaggcgagttcagcgaccgcgccatcgcctatatgctgacccaggacgaggcccaggggccgttctaccgtcaatcctggggcggcatgaaggcctgtacgccgatcatcagcggcggcatgaatgccctgcgcatgcccggctttttcgagaacctgggcaacgccaatgtcatcctgaccgccggcggcggcgccttcggccatatcgacggcccggtggccggggcgcggtcgttgcgtcaagcctggcaagcctggcgtgatggggttccggttctggattatgcccgcgagcacaaggaactggcccgcgccttcgagtccttccccggcgacgccgaccagatctatccgggctggcgcaaggccctgggcgtcgaggacacccgcagcgcccttccggcgtaa

SEQ ID NO: 16 - protein, 466 aa, Rhodospirillum rubrum
MDQSSRYVNLALKEEDLIAGGEHVLCAYILKPKPGYGYVATAAHFAAESSTGTNVEVCTTDDFTRGVDALVYEVDEARELTKIAYPVALFDRNITDGKAMIASFLTLTMGNNQGMGDVEYAKMHDFYVPDSYRALFDGPSVNISALWKVLGRPEVDGGLVVGTIIKPKLGLRPKPFAEACHAFWLGGDFIKNDEPQGNQPFAPLRDTIALVADAMKRAQDETGEAKLFSANITADDPFEIIARGEYVLKTFGENASHVALLVDGYVAGAAAITTARRRFPDNFLHYHRAGHGAVTSPQSKRGYTAFVHCKMARLQGASGIQTGTMGFGKMEGEFSDRAIAYMLTQDEAQGPFYRQSWGGMKACTPIISGGMNALRMPGFFENLGNANVILTAGGGAFGHIDGPVAGARSLRQAWQAWRDGVPVLDYAREHKELARAFESFPGDADQIYPGWRKALGVEDTRSALPA

SEQ ID NO: 17 - DNA, 1401 bp, Rhodospirillum rubrum
atggaccagtcatctcgttacgtcaatctggcgctcaaggaagaggatctgatcgctggcggcgagcatgtgctctgtgcctatatcctgaagcccaagcccggatatggctatgtggcgaccgcggcgaatttcgccgccgagagttcgacgggcaccaacgtcgaggtttgcaccaccgatgatttcacccggggcgtcgacgccctggtctatgaggtggacgaggcccgcgagctgaccaagatcgcctatccggtggctttgttcgaccgcaacatcaccgacggcaaggcgatgatcgcctcgttcctgacgctcaccatgggaaataaccagggtatgggcgacgtggaatacgccaagatgcacgatttctatgtgcccgactcctatcgcgccctgttcgatggccccagcgtcaatatctcggccctgtggaaggtgctaggtcggcccgaggtcgacggcggtttggtcgtcggcacgatcatcaagccgaagctcggcctgcgtcccaagcccttcgccgaggcctgccacgccttctggctgggcggcgatttcatcaagaacgacgagccccagggcaatcagcccttcgcccccttgcgcgacaccatcgccctggtcgccgacgccatgaagcgggcccaggacgagaccggcgaggccaagctgttctcggccaatatcaccgccgacgatcccttcgagatcatcgcgcgtggcgagtatgtgctgaagaccttcggcgagaacgcctcgcatgtcgccttgctggtcgacggctatgtcgccggcgccgcggcgatcaccacggcgcgccgccgtttccccgataacttcctgcattatcaccgggccggccacggcgccgtcacctcgccccagtccaagcgcggctataccgccttcgtccattgcaagatggcccgcctgcagggcgccagcggcatccacaccggcaccatgggctttggcaagatggaaggcgagtccagcgaccgcgccatcgcctatatgctgacccaggacgaggcccaggggccgttctaccgtcaatcctggggcggcatgaaggcctgtacgccgatcatcagcggcggcatgaatgccctgcgcatgcccggctttttcgagaacctgggcaacgccaatgtcatcctgaccgccggcggcggcgccttcggccatatcgacggcccggtggccggggcgcggtcgttgcgtcaagcctggcaagcctggcgtgatggggttccggttctggattatgcccgcgagcacaaggaactggcccgcgccttcgagtccttccccggcgacgccgaccagatctatccgggctggcgcaaggccctgggcgtcgaggacacccgcagcgcccttccggcgtaa

SEQ ID NO: 18 - protein, 466 aa, Rhodospirillum rubrum
MDQSSRYVNLALKEEDLIAGGEHVLCAYILKPKPGYGYVATAANFAAESSTGTNVEVCTTDDFTRGVDALVYEVDEARELTKIAYPVALFDRNITDGKAMIASFLTLTMGNNQGMGDVEYAKMHDFYVPDSYRALFDGPSVNISALWKVLGRPEVDGGLVVGTIIKPKLGLRPKPFAEACHAFWLGGDFIKNDEPQGNQPFAPLRDTIALVADAMKRAQDETGEAKLFSANITADDPFEIIARGEYVLKTFGENASHVALLVDGYVAGAAAITTARRRFPDNFLHYHRAGHGAVTSPQSKRGYTAFVHCKMARLQGASGIHTGTMGFGKMEGESSDRAIAYMLTQDEAQGPFYRQSWGGMKACTPIISGGMNALRMPGFFENLGNANVILTAGGGAFGHIDGPVAGARSLRQAWQAWRDGVPVLDYAREHKELARAFESFPGDADQIYPGWRKALGVEDTRSALPA

SEQ ID NO: 19 - DNA, 1326 bp, Archaeoglobus fulgidus
atggcggagtttgagatttacagagagtatgttgacaaaagctacgagccgcagaaggatgacatcgttgcagttttcaggataactcccgccgagggcttcactattgaggacgccgccggcgctgttgcggcggagagcagcacggggacttggacatcccttcatccatggtatgatgaggaaagggttaaggggctttcagcaaaggcttacgatttcgtggatttgggtgatggtagcagcatagtcagaattgcttacccatctgagcttttcgagccccacaacatgccgggtttgcttgcctctattgccggaaacgttttcggcatgaagagggtgaaggggctgaggcttgaggatttgcagctgccaaagtccttcctcaaggacttcaaggggccatcgaagggaaaggagggtgtaaagaaaatttttggtgttgcagacaggcccattgtcgggactgtgccaaagccgaaggttggctactctgcagaggaggttgaaaagctggcttacgagcttctttcaggcgggatggactacatcaaggatgacgagaacctcacaagtcctgcatactgcagattcgaggagagggcggagaggataatgaaggtcatcgagaaggttgaggctgaaacgggcgagaagaagtcttggttcgccaacatcaccgcagatgtgagggagatggagaggaggctgaagcttgtagctgaactcggcaatccgcacgttatggttgatgtcgtaataaccggctggggggcgcttgagtacatcagagaccttgcggaggattacgatttggccatacacggccatagagctatgcatgcagccttcacccgcaatgctaagcacggcatatcgatgttcgttctggcaaagctctaccgcataatcggcatcgaccagctccacatagggactgcaggagcgggaaagcttgaggggcagaagtgggacaccgtgcagaacgcgagaattttcagcgaggttgagtacactccagatgagggcgacgcattccatctcagccagaacttccaccacataaagccggcgatgcccgtttcatctggcggactgcatccaggaaatctggagccggtaatcgacgccctcggcaaggagatagtcattcaggttggtgggggagttcttggccacccgatgggagcaaaggcgggggcaaaggctgtgaggcaggctcttgatgccatcatctctgcaatcccgctggaggagcatgcaaagcagcatccggagctacaggccgctctggagaagtggggcagggttacgccaatctaa

SEQ ID NO: 20 - protein, 441 aa, Archaeoglobus fulgidus
MAEFEIYREYVDKSYEPQKDDIVAVFRITPAEGFTIEDAAGAVAAESSTGTWTSLHPWYDEERVKGLSAKAYDFVDLGDGSSIVRIAYPSELFEPHNMPGLLASIAGNVFGMKRVKGLRLEDLQLPKSFLKDFKGPSKGKEGVKKIFGVADRPIVGTVPKPKVGYSAEEVEKLAYELLSGGMDYIKDDENLTSPAYCRFEERAERIMKVIEKVEAETGEKKSWFANITADVREMERRLKLVAELGNPHVMVDVVITGWGALEYIRDLAEDYDLAIHGHRAMHAAFTRNAKHGISMFVLAKLYRIIGIDQLHIGTAGAGKLEGQKWDTVQNARIFSEVEYTPDEGDAFHLSQNFHHIKPAMPVSSGGLHPGNLEPVIDALGKEIVIQVGGGVLGHPMGAKAGAKAVRQALDAIISAIPLEEHAKQHPELQAALEKWGRVTPI

SEQ ID NO: 21 - DNA, 2376 bp, Synechocystis PCC6803
atgcccaagacgcaatctgccgcaggctataaggccggggtgaaggactacaaactcacctattacacccccgattacacccccaaagacactgacctgctggcggctttccgcttcagccctcagccgggtgtccctgctgacgaagctggtgcggcgatcgcggctgaatcttcgaccggtacctggaccaccgtgtggaccgacttgctgaccgacatggatcggtacaaaggcaagtgctaccacatcgagccggtgcaaggcgaagagaactcctactttgcgttcatcgcttacccgctcgacctgtttgaagaagggtcggtcaccaacatcctgacctcgatcgtcggtaacgtgtttggcttcaaagctatccgttcgctgcgtctggaagacatccgcttccccgtcgccttggtcaaaaccttccaaggtcctccccacggtatccaagtcgagcgcgacctgctgaacaagtacggccgtccgatgctgggttgcacgatcaaaccaaaactcggtctgtcggcgaaaaactacggtcgtgccgtctacgaatgtctgcgcggcggtctggacttcaccaaagacgacgaaaacatcaactcgcagccgttccaacgctggcgcgatcgcttcctgtttgtggctgatgcaatccacaaatcgcaagcagaaaccggtgaaatcaaaggtcactacctgaacgtgaccgcgccgacctgcgaagaaatgatgaaacgggctgagttcgctaaagaactcggcatgccgatcatcatgcatgacttcttgacggctggtttcaccgccaacaccaccttggcaaaatggtgccgcgacaacggcgtcctgctgcacatccaccgtgcaatgcacgcggtgatcgaccgtcagcgtaaccacgggattcacttccgtgtcttggccaagtgtttgcgtctgtccggtggtgaccacctccactccggcaccgtcgtcggcaaactggaaggcgacaaagcttcgaccttgggctttgttgacttgatgcgcgaagaccacatcgaagctgaccgcagccgtggggtcttcttcacccaagattgggcgtcgatgccgggcgtgctgccggttgcttccggtggtatccacgtgtggcacatgcccgcactggtggaaatcttcggtgatgactccgttctccagttcggtggcggcaccttgggtcacccctggggtaatgctcctggtgcaaccgcgaaccgtgttgccttggaagcttgcgtccaagctcggaacgaaggtcgcgacctctaccgtgaaggcggcgacatccttcgtgaagctggcaagtggtcgcctgaactggctgctgccctcgacctctggaaagagatcaagttcgaattcgaaacgatggacaagctctaaggagcctctgactatcgctgggggagtgagcgttgctgcgtaaagctttctccccagcctttcgacttaacctttcaggatttctgaatcatgagcatgaaaactctgcccaaagagcgtcgtttcgagactttctcgtacctgcctcccctcagcgatcgccaaatcgctgcacaaatcgagtacatgatcgagcaaggcttccaccccttgatcgagttcaacgagcactcgaatccggaagagttctactggacgatgtggaagctccccctgtttgactgcaagagccctcagcaagtcctcgatgaagtgcgtgagtgccgcagcgaatacggtgattgctacatccgtgtcgctggcttcgacaacatcaagcagtgccaaaccgtgagcttcatcgttcatcgtcccggccgctactaaggatcctactgacctaggtcacactggctcaccttcgggtgggcctttctgcgtttatatactagagagagaatataaaaagccagattattaatccggcttttttattattttactagtatgcaaactaagcacatagctcaggcaacagtgaaagtactgcaaagttacctcacctaccaagccgttctcaggatccagagtgaactcggggaaaccaaccctccccaggccatttggttaaaccagtatttagccagtcacagtattcaaaatggagaaacgtttttgacggaactcctggatgaaaataaagaactggtactcaggatcctggcggtaagggaagacattgccgaatcagtgttagattttttgcccggtatgacccggaatagcttagcggaatctaacatcgcccaccgccgccatttgcttgaacgtctgacccgtaccgtagccgaagtcgataatttcccttcggaaacctccaacggagaatcaaacaacaacgattctcccccgtcctaa

SEQ ID NO: 22 - protein, 469 aa, Synechocystis PCC6803
MVQAKAGFKAGVQDYRLTYYTPDYTPKDTDLLACFRMTPQPGVPAEEAAAAVAAESSTGTWTTVWTDNLTDLDRYKGRCYDLEAVPNEDNQYFAFIAYPLDLFEEGSVTNVLTSLVGNVFGFKALRALRLEDIRFPVALIKTFQGPPHGITVERDKLNKYGRPLLGCTIKPKLGLSAKNYGRAVYECLRGGLDFTKDDENINSQPFMRWRDRFLFVQEAIEKAQAETNEMKGHYLNVTAGTCEEMMKRAEFAKEIGTPIIMHDFFTGGFTANTTLARWCRDNGILLHIHRAMHAVVDRQKNHGIHFRVLAKCLRLSGGDHLHSGTVVGKLEGERGITMGFVDLMREDYVEEDRSRGIFFTQDYASMPGTMPVASGGIHVWHMPALVEIFGDDSCLQFGGGTLGHPWGNAPGATANRVALEACVQARNEGRNLAREGNDVIREACRWSPELAAACELWKEIKFEFEAMDT

SEQ ID NO: 23 - protein, 136 aa, Synechocystis PCC6803
MQTKHIAQATVKVLQSYLTYQAVLRIQSELGETNPPQAIWLNQYLASHSIQNGETFLTELLDENKELVLRILAVREDIAESVLDFLPGMTRNSLAESNIAHRRHLLERLTRTVAEVDNFPSETSNGESNNNDSPPS

SEQ ID NO: 24 - protein, 113 aa, Synechocystis PCC6803
MKTLPKERRYETLSYLPPLTDQQIAKQVEFLLDQGFIPGVEFEEDPQPETHFWTMWKLPFFGGATANEVLAEVRECRSENPNCYIRVIGFDNIKQCQTVSFIVHKPNQNQGRY

SEQ ID NO: 25 - DNA, 1302 bp, Escherichia coli
atgaaaacccgtacacaacaaattgaagaattacagaaagagtggactcaaccgcgttgggaaggcattactcgcccatacagtgcggaagatgtggtgaaattacgcggttcagtcaatcctgaatgcacgctggcgcaactgggcgcagcgaaaatgtggcgtctgctgcacggtgagtcgaaaaaaggctacatcaacagcctcggcgcactgactggcggtcaggcgctgcaacaggcgaaagcgggtattgaagcagtctatctgtcgggatggcaggtagcggcggacgctaacctggcggccagcatgtatccggatcagtcgctctatccggcaaactcggtgccagctgtggtggagcggatcaacaacaccttccgtcgtgccgatcagatccaatggtccgcgggcattgagccgggcgatccgcgctatgtcgattacttcctgccgatcgttgccgatgcggaagccggttttggcggtgtcctgaatgcctttgaactgatgaaagcgatgattgaagccggtgcagcggcagttcacttcgaagatcagctggcgtcagtgaagaaatgcggtcacatgggcggcaaagttttagtgccaactcaggaagctattcagaaactggtcgcggcgcgtctggcagctgacgtgacgggcgttccaaccctgctggttgcccgtaccgatgctgatgcggcggatctgatcacctccgattgcgacccgtatgacagcgaatttattaccggcgagcgtaccagtgaaggcttcttccgtactcatgcgggcattgagcaagcgatcagccgtggcctggcgtatgcgccatatgctgacctggtctggtgtgaaacctccacgccggatctggaactggcgcgtcgctttgcacaagctatccacgcgaaatatccgggcaaactgctggcttataactgctcgccgtcgttcaactggcagaaaaacctcgacgacaaaactattgccagcttccagcagcagctgtcggatatgggctacaagttccagttcatcaccctggcaggtatccacagcatgtggttcaacatgtttgacctggcaaacgcctatgcccagggcgagggtatgaagcactacgttgagaaagtgcagcagccggaatttgccgccgcgaaagatggctataccttcgtatctcaccagcaggaagtgggtacaggttacttcgataaagtgacgactattattcagggcggcacgtcttcagtcaccgcgctgaccggctccactgaagaatcgcagttc

SEQ ID NO: 26 - protein, 434 aa, Escherichia coli
MKTRTQQIEELQKEWTQPRWEGITRPYSAEDVVKLRGSVNPECTLAQLGAAKMWRLLHGESKKGYINSLGALTGGQALQQAKAGIEAVYLSGWQVAADANLAASMYPDQSLYPANSVPAVVERINNTFRRADQIQWSAGIEPGDPRYVDYFLPIVADAEAGFGGVLNAFELMKAMIEAGAAAVHFEDQLASVKKCGHMGGKVLVPTQEAIQKLVAARLAADVTGVPTLLVARTDADAADLITSDCDPYDSEFITGERTSEGFFRTHAGIEQAISRGLAYAPYADLVWCETSTPDLELARRFAQAIHAKYPGKLLAYNCSPSFNWQKNLDDKTIASFQQQLSDMGYKFQFITLAGIHSMWFNMFDLANAYAQGEGMKHYVEKVQQPEFAAAKDGYTFVSHQQEVGTGYFDKVTTIIQGGTSSVTALTGSTEESQF

SEQ ID NO: 27 - DNA, 1287 bp, Mycobacterium tuberculosis
atgtccgtggtgggtacccctaaatccgccgaacaaatccaacaagaatgggacaccaatccccgttggaaagatgttacccggacctactccgccgaagatgtagtcgctctccaaggtagcgtcgtcgaagaacacaccctggcgcggcggggagccgaagtgttgtgggaacaattgcatgatttagaatgggtgaatgctttgggtgccttgaccggtaacatggccgtgcaacaagttcgtgccggtctgaaagccatttatttgtccggttggcaagtggccggtgatgccaatttgagtggtcacacctaccccgatcagtccttgtaccccgccaattccgtgccccaagtggtgcggcgtattaataacgccttgcaacgggccgatcaaattgccaaaattgaaggcgatacctccgtggaaaattggttggcccctattgtggccgatggtgaagccggttttggtggcgccttgaatgtgtatgaattgcaaaaggccctgattgccgccggtgtggccggttcccactgggaagatcaattggcctccgaaaagaaatgtggtcatttgggtggtaaagtgctgattcccacccaacaacatattcgcactttgacctccgcccggctggccgcagatgtggccgatgtgcctaccgtggtgattgcccgtaccgatgccgaagccgccaccttgattacctccgatgttgatgaacgtgatcaaccctttattaccggtgaacggactcgggaaggcttttacaggaccaagaacggcattgaaccctgtattgcccgcgccaaagcctatgctccttttgccgacttgatttggatggaaaccggcactcccgacttggaagcggcccggcaatttagtgaagccgttaaagccgaataccccgatcaaatgttggcctacaattgtagtccctcctttaattggaagaaacatttagatgatgccaccattgccaaatttcagaaagaattggccgctatgggttttaaatttcaatttattaccttggccggttttcacgccttgaattactccatgtttgatttggcctacggttatgcccaaaatcaaatgtccgcctatgttgaattgcaagaacgtgaatttgccgccgaagaacgtggttacaccgccaccaaacatcaacgtgaagtgggtgccggctattttgatcgtattgccaccaccgtggaccccaattcctccaccaccgccttgaccggttccaccgaagaaggtcaatttcattaa

SEQ ID NO: 28 - protein, 428 aa, Mycobacterium tuberculosis
MSVVGTPKSAEQIQQEWDTNPRWKDVTRTYSAEDVVALQGSVVEEHTLARRGAEVLWEQLHDLEWVNALGALTGNMAVQQVRAGLKAIYLSGWQVAGDANLSGHTYPDQSLYPANSVPQVVRRINNALQRADQIAKIEGDTSVENWLAPIVADGEAGFGGALNVYELQKALIAAGVAGSHWEDQLASEKKCGHLGGKVLIPTQQHIRTLTSARLAADVADVPTVVIARTDAEAATLITSDVDERDQPFITGERTREGFYRTKNGIEPCIARAKAYAPFADLIWMETGTPDLEAARQFSEAVKAEYPDQMLAYNCSPSFNWKKHLDDATIAKFQKELAAMGFKFQFITLAGFHALNYSMFDLAYGYAQNQMSAYVELQEREFAAEERGYTATKHQREVGAGYFDRIATTVDPNSSTTALTGSTEEGQFH

SEQ ID NO: 29 - DNA, 870 bp, Arabidopsis thaliana
atggaagtgggtttcttaggcttgggtattatgggtaaagccatgtccatgaacttgttgaagaacggctttaaagtaaccgtttggaaccgcaccttgtccaaatgcgacgaacttgtcgaacacggagccagcgtgtgcgaaagtcccgccgaagttattaagaaatgtaaatacaccattgccatgctgtccgatccctgtgccgccttgtccgtggtgtttgataaaggcggtgtgctggaacaaatttgtgaaggcaaaggctacatcgatatgtccaccgtggatgccgaaacctccttgaaaattaacgaagccatcaccggtaaaggcggacgttttgtggaaggccccgtgtccggttccaagaaacccgccgaagatggtcaattgatcattttggccgccggcgacaaagccttgtttgaagaaagtattcccgcctttgatgtgttgggtaaacgttctttctacttgggtcaagtgggtaacggtgccaaaatgaaattgattgtgaacatgatcatgggttccatgatgaacgcctttagtgaaggtttggtgttggccgataaatccggtttgtcctccgataccttgttggacattttggacttgggcgccatgaccaaccccatgtttaaaggtaaaggcccctccatgaacaaatcctcctaccctcccgcctttcccctgaaacaccaacagaaagatatgcggttggccttggccctgggtgatgaaaacgccgtgtccatgcccgtggccgccgctgccaacgaagccttcaagaaagcccggtccttgggcttgggtgatttggattttagcgccgtgattgaagccgtgaaattttcccgtgaataa

SEQ ID NO: 30 - protein, 289 aa, Arabidopsis thaliana
MEVGFLGLGIMGKAMSMNLLKNGFKVTVWNRTLSKCDELVEHGASVCESPAEVIKKCKYTIAMLSDPCAALSVVFDKGGVLEQICEGKGYIDMSTVDAETSLKINEAITGKGGRFVEGPVSGSKKPAEDGQLIILAAGDKALFEESIPAFDVLGKRSFYLGQVGNGAKMKLIVNMIMGSMMNAFSEGLVLADKSGLSSDTLLDILDLGAMTNPMFKGKGPSMNKSSYPPAFPLKHQQKDMRLALALGDENAVSMPVAAAANEAFKKARSLGLGDLDFSAVIEAVKFSRE

SEQ ID NO: 31 - DNA, 1002 bp, Synechococcus elongatus
atgagcaagccagatcgtgttgttttgatcggcgttgccggtgactccggttgcggcaaatcaaccttcctaaatcgccttgccgacttgtttggtacggaattgatgacggtcatctgcttggatgactatcacagtctcgatcgcaagggccggaaggaagcaggcgtaacggctttggatccccgcgccaacaactttgacttgatgtatgaacaggtcaaggcgttgaagaacggcgaaacgatcatgaagccgatctacaaccatgaaaccggcttgatcgatccgcccgaaaaaatcgaacccaatcgcatcattgtgatcgagggtctgcatccgctttacgacgagcgcgtgcgtgaactgctcgatttcagcgtttacctcgacatcgatgacgaagtcaaaatcgcttggaagatccaacgcgatatggcagaacgcggccactcctacgaagatgtcctcgcctcgatcgaagcgcgccgccctgacttcaaggcctacattgagccccagcgtggccatgcggacatcgtcatccgcgtcatgccgacccagctaatccccaatgacaccgagcgcaaggtgctgcgggtgcagttgatccaacgggaaggccgcgatggttttgagccggcttacctgttcgacgaaggttcgaccatccagtggacgccctgcggtcgtaagctgacctgctcctatccgggcattcgcttagcctacggccctgacacctactacggtcacgaagtctcagtgcttgaggtcgacggtcagttcgagaacctcgaagagatgatctacgtcgagggccacctcagcaagaccgacacgcagtactacggtgagttgacccacctgctgctgcagcacaaagattacccgggttcgaacaacggcacgggtctgttccaagtgctgaccggcctgaaaatgcgggcggcctatgagcgtttgacctcccaagcagcacccgtcgccgctagcgtctag

SEQ ID NO: 32 - protein, 333 aa, Synechococcus elongatus
MSKPDRVVLIGVAGDSGCGKSTFLNRLADLFGTELMTVICLDDYHSLDRKGRKEAGVTALDPRANNFDLMYEQVKALKNGETIMKPIYNHETGLIDPPEKIEPNRIIVIEGLHPLYDERVRELLDFSVYLDIDDEVKIAWKIQRDMAERGHSYEDVLASIEARRPDFKAYIEPQRGHADIVIRVMPTQLIPNDTERKVLRVQLIQREGRDGFEPAYLFDEGSTIQWTPCGRKLTCSYPGIRLAYGPDTYYGHEVSVLEVDGQFENLEEMIYVEGHLSKTDTQYYGELTHLLLQHKDYPGSNNGTGLFQVLTGLKMRAAYERLTSQAAPVAASV

SEQ ID NO: 33 - DNA, 2604 bp, Artificial Sequence
  PBRS-mazF cassette polynucleotide
gtcgactttgacggcgtaaagttgataaaatagaattaagaatggactatcggtacagaaaaaatgggtaactggatggtgaataaacttcccttacccaatgcactctccaccgttaaagaccccctatgcttaacggtgatcacctgggcaatggcgagtcccaaccctgtcccccccgttttgcgcgaacgatctcgattaactcggtaaaaacgctcaaaaatgtgttcctgttggtcgggggcaatgccgatgccggtatcttgcacggtgatgatagccatctgttcatgggatgtcagggtaatatcaacacgtcccccagcagttgtgtattgaatggcgttggcaattaggtttgagaccagtcgatagagttgggattcattaccccaggcgtaaacttcccctgaactcagatcactgctgagatcaatgtgggcggcgatcgctaattctaaaaactcttcggtgaggtcactgactaaatcatttaaacaacaaagccgccaatcttcggcggtggtttcctgctctaagcgacttagtagcaataaatccgtaatcaattggcttaatcgccttccctgtcgttcaacggtatgtagcatggtgttaatttctggggaatggcttgagtcgatgcgtaataccgcttccaccgtggccaacagactagccaatggcgatcgtaattcatgggctgcattcgcggtgaattgttgttgttgttggtaggactggtaaatgggacgcatggctaaccccgctaagccccaactggagaaggcgaccaaacccagggcaatgggaaaactaagccctaaaatccaaagaatacgtttattttcggcatcaaaggctgccaggctccggccaatttgtagatagccccaggaagatttgtctgtattaccggcgctatgcaaaatggtggtgaattgtcgataccgatcgccggttggggggtgaatagtctgccaagtttcctggttaaaaatggaggatagggaagccggttgattaggcgaaaaagccagcaggttgccttgataatcaaataaacgaatgtaatataaactgcgatcactaatgcccaacgtgtgacgttcaatcagggtggggttgacctggcagggttggttgaccaaacacagatcgggcaacattttttgtaatactccggtgggactagcattactcggcaacatcggctctaaactgtcatgcaacgtcccggcgatcgactccacttctcgctccaacgccatccagttggcctgcacaatggcacgataaacccccaaccccaacagggtaagaattccccccattactagggcataccagaaagccaattgcagacgactacgggcaaagaggcgacgggtattcatggcgatagggtgaaccgatagccttgaccgggaactgttttaattgggcaaggacaattttgttgagctagcttgcgtcgtatcaaacgcatttgggccgccaccacattactcatgggctcctcatcaagatcccacagttgttgccggatcttgctaccggaaatgatccgctctgggttttgcatcagatattgaaaaatttgaaattctcttacggttaaagcaatttcctgtctttctaggtttagtggctccgagatagttaccgataacagattattactgggatcaaggctgaagttgcccaaagttaaaatttgcggttggaattgtggcgatcgccgttgtagtgcccgcagtcttgctaatagctctgccatcacaaacggttttgttagatagtcatctgccccggcatctagtccttcgacacggttttccggttctcctaacgctgttaacatcaacaccggcaaggaattaccctgggttctcagtttttgacagagttccaaacccgataatcccggcagtaaccaatccacaatggcaagggtgtattccgtccattgattttccaaataatcccaagcttgggagccatccgtcacccaatccaccacatacttttcactaactagcactttcttaatagccattcccaaatccgtctcatcttccaccagcaaaattcgcatcgcctctgccttttttataacggtctgatcttagcgggggaaggagattttcacctgaatttcataccccctttggcagactgggaaaatcttggacaaattcccaatttgaggtggtcatatggtaagccgatacgtacccgatatgggcgatctgatttgggttgattttgacccgacaaaaggtagcgagcaagctggacatcgtccagctgttgtcctgagtcctttcatgtacaacaacaaaacaggtatgtgtctgtgtgttccttgtacaacgcaatcaaaaggatatccgttcgaagttgttttatccggtcaggaacgtgatggcgtagcgttagctgatcaggtaaaaagtatcgcctggcgggcaagaggagcaacgaagaaaggaacagttgccccagaggaattacaactcattaaagccaaaattaacgtactgattgggtagtgttactaactcacttctattctggtcacaggttactagtctgcag

SEQ ID NO: 34 - DNA, 2064 bp, Synechocystis PCC6803
gtgctggcaaagtccctgggctggttgttggctgtatctaggaggaactattgcatgggatctcgcaccgccttggcttccaggccttggtccaaacatttagcagacccccagattgacccgacggcctacgtgcattcgtttgccaatgtggtgggggatgtgcgcattcagcccggggtcagtgttgcccctgggagctccattcgagcggacgagggcacgcccttttggattggcggtaatgtgctgatccaacatggagtggtgatccatggcttagaaactggtcgggtgctgggggatgatgaccaggaatattctgtttggattgggccgggcacctgtgtggcccatttggctttggtccacggcccagtttacctcggcgctaattgcttcattggtttccgttccactgtgctcaatgcacgggtgggggatggggcggtggtgatgatgcattccctagttcaggatgtggaaattccccccaacaaattggtgccctccggtgccatgattacccagcaacaccaggcggatagtttaccggatgtgcaagctggcgatcgccattttgtccagcagattgcggccatgcacggacaaagtgcttctccaacccagggaactgatccaaccgtgtgtgtgttgccggagtccctccccgccgttacccccgttactgaaaccccctatataaattccatagacaacatgagtattaattctgacattaccaaccagatccgctccctcctggcccagggctatggtatcggggcggaacacgccaacgaacgacgtttcaaaaccaaatcttggcagagctgtggcaccgccgatggtttccgtcccgaccaggttattgccacggtggaaggttggctccaggagtttgcgggggaatatgtccgcctcattggcattgaccagggggctaaacgccgggtagtggaagtgattattcaacgccccggtgatgttcccggttctcctagtcggggtaccaccaccaccaaagccctaagcagtggcggtagcggccggagtgcggtggcccaccaaacaggtaacttagctggggatagtgctaaccagttgcgggccctgttgcatcagggttataaaatcggcttggaatatgccagtgcccgtcgcttcaaaaccggctcttggttaactggaggcaccattggtagtcatcgggaaggggaagctttgcaggaattaaatcgtttcctggccgaccacaccaatgagtatgtgcgcattatcggtattgatccagccggtaagcggcgggtggcagaaattgttgtacaccgtcccaatggtaatggcaatggtaaaccttctagttccagcagttccgttggctataagtctgcccctgtgagctccgccgggggctctagtgctggtggtttaaccccagaagtgatagcaacggtgcggggattactcgccaacggccatagcattggtaccgaacacacagataaacgtcgctttaaggccaaatcctgggatacttgtcccaccattgatggtggccgggaagctgaagttttagccaaattggaagcttgcctagcagatcatgccggggagtatgtgcggattattggtattgaccgggttggtaagcgacgggtgttagaacagattattcaacgtccaggggacaacgttgttgctgggcgatcgccgtcttcgtctagtgctagtacatccagtagtgcctccagcaatggctttggcagtggcaatggtggtggttacagtaattctgccgtgcgcctagataacagcgtggttacccaggtgcgttccctcctggcccagggttacaaaattggcaccgaacacacggataaacgtcgctttaaagccaaatcctggcagagttgtgcccctatcaccagtacccacgagtcggaagtgttgcgggccctagaaggttgtttggcagaccataacggcgaatatgtccgcttactaggcatagatcccacggctaaacggcgggtgttggaaaccattattcagcgtccctag

SEQ ID NO: 35 - DNA, 6176 bp, Artificial Sequence
  pHKH-RFP polynucleotide
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccctttgacaacaatgtggcctggaataacctgggggatttgtccaccaccacccaacgggcctacacttcggctattagcacagacacagtgcagagtgtttatggcgttaatctggaaaaaaacgataacattcccattgtttttgcgtggcccatttttcccaccacccttaatcccacagattttcaggtaatgcttaacacgggggaaattgtcaccccggtgatcgcctctttgattcccaacagtgaatacaacgaacggcaaacggtagtaattacgggcaattttggtaatcgtttaaccccaggcacggagggagcgatttatcccgtttccgtaggcacagtgttggacagtactcctttggaaatggtgggacccaacggcccggtcagtgcggtgggtattaccattgatagtctcaacccctacgtggccggcaatggtcccaaaattgtcgccgctaagttagaccgcttcagtgacctgggggaaggggctcccctctggttagccaccaatcaaaataacagtggcggggatttatatggccgcgggaattcgcggccgcttctagagttgacaattaatcatccggctcgtataatgtgtggaattgtgagcggataacaatttcacacagctagcattaaagaggagaaatgacatatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataacgctgatagtgctagtgtagatcgctacggatcctactgacctaggtcacactggctcaccttcgggtgggcctttctgcgtttatatactagagagagaatataaaaagccagattattaatccggcttttttattattttactagtagcggccgctgcaggatatcaagcttatcgataccgtcgacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcggtcgacctcgagagaccaagcccaatttcgtttgcgaatttacaccagcgccggtttttcccccgatggcattgccagtttactacccacagaatttgaacggtattttcaactccaagcggaagatattacgggacggacagttatcctaacccaaactggtgttgattatgaaattcccggctttggtctggtgcaggtgttggggctggcggatttggccggggttcaggacagctatgacctgacttacatcgaagatcatgacaactattacgacattatcctcaaaggggacgaagccgcagttcgccaaattaagagggttgctttgccctccgaaggggattattcggcggtttataatcccggtggccccggcaatgatccagagaatggtcccccagggccctttactgtgtccagtagtccccaggtaattaaggtaacggataccatcggccagcccaccaaagtctcctatgtggaagtggatggccccgtattgcgtaatcccttcagtggtactcccattgggcaagaggtgggtttagcggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 36 - DNA, 7157 bp, Artificial Sequence
  pAVO+ (RSF1010)
gatctgtaatccgggcagcgcaacggaacattcatcagtgtaaaaatggaatcaataaagccctgcgcagcgcgcagggtcagcctgaatacgcgtatgccgggagtgtacacacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacggatatcgagctcgcttgacgcgtattcaggctgaccctgcgcgctgcgcagggctttattgattccatttttacactgatgaatgttccgttgcgctgcccggattacagctgaaagcgaccaggtgctcggcgtggcaagactcgcagcgaacccgtagaaagccatgctccagccgcccgcattggagaaattcttcaaattcccgttgcacatagcccggcaattcctttccctgctctgccataagcgcagcgaatgccgggtaatactcgtcaacgatctgatagagaagggtttgctcgggtcggtggctctggtaacgaccagtatcccgatcccggctggccgtcctggccgccacatgaggcatgttccgcgtccttgcaatactgtgtttacatacagtctatcgcttagcggaaagttcttttaccctcagccgaaatgcctgccgttgctagacattgccagccagtgcccgtcactcccgtactaactgtcacgaacccctgcaataactgtcacgcccccctgcaataactgtcacgaacccctgcaataactgtcacgcccccaaacctgcaaacccagcaggggcgggggctggcggggtgttggaaaaatccatccatgattatctaagaataatccactaggcgcggttatcagcgcccttgtggggcgctgctgcccttgcccaatatgcccggccagaggccggatagctggtctattcgctgcgctaggctacacaccgccccaccgctgcgcggcagggggaaaggcgggcaaagcccgctaaaccccacaccaaaccccgcagaaatacgctggagcgcttttagccgctttagcggcctttccccctacccgaagggtgggggcgcgtgtgcagccccgcagggcctgtctcggtcgatcattcagcccggctcatccttctggcgtggcggcagaccgaacaaggcgcggtcgtggtcgcgttcaaggtacgcatccattgccgccatgagccgatcctccggccactcgctgctgttcaccttggccaaaatcatggcccccaccagcaccttgcgccttgtttcgttcttgcgctcttgctgctgttcccttgcccgcacccgctgaatttcggcattgattcgcgctcgttgttcttcgagcttggccagccgatccgccgccttgttgctccccttaaccatcttgacaccccattgttaatgtgctgtctcgtaggctatcatggaggcacagcggcggcaatcccgaccctactttgtaggggagggcgcacttaccggtttctcttcgagaaactggcctaacggccacccttcgggcggtgcgctctccgagggccattgcatggagccgaaaagcaaaagcaacagcgaggcagcatggcgatttatcaccttacggcgaaaaccggcagcaggtcgggcggccaatcggccagggccaaggccgacttcatccagcgcgaaggcaagtatgcccgcgacatggatgaagtcttgcacgccgaatccgggcacatgccggagttcgtcgagcggcccgccgactactgggatgctgccgacctgtatgaacgcgccaatgggcggctgttcaaggaggtcgaatttgccctgccggtcgagctgaccctcgaccagcagaaggcgctggcgtccgagttcgcccagcacctgaccggtgccgagcgcctgccgtatacgctggccatccatgccggtggcggcgagaacccgcactgccacctgatgatctccgagcggatcaatgacggcatcgagcggcccgccgctcagtggttcaagcggtacaacggcaagaccccggagaagggcggggcacagaagaccgaagcgctcaagcccaaggcatggcttgagcagacccgcgaggcatgggccgaccatgccaaccgggcattagagcgggctggccacgacgcccgcattgaccacagaacacttgaggcgcagggcatcgagcgcctgcccggtgttcacctggggccgaacgtggtggagatggaaggccggggcatccgcaccgaccgggcagacgtggccctgaacatcgacaccgccaacgcccagatcatcgacttacaggaataccgggaggcaatagaccatgaacgcaatcgacagagtgaagaaatccagaggcatcaacgagttagcggagcagatcgaaccgctggcccagagcatggcgacactggccgacgaagcccggcaggtcatgagccagacccagcaggccagcgaggcgcaggcggcggagtggctgaaagcccagcgccagacaggggcggcatgggtggagctggccaaagagttgcgggaggtagccgccgaggtgagcagcgccgcgcagagcgcccggagcgcgtcgcgggggtggcactggaagctatggctaaccgtgatgctggcttccatgatgcctacggtggtgctgctgatcgcatcgttgctcttgctcgacctgacgccactgacaaccgaggacggctcgatctggctgcgcttggtggcccgatgaagaacgacaggactttgcaggccataggccgacagctcaaggccatgggctgtgagcgcttcgatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtagtgcttgcgttggtactcacgcctgttatactatgagtactcacgcacagaagggggttttatggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactgatatgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaaccaccaactgcctgcaaaggaggcaatcaatggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgatgccatggtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgccagcgggtgcgcgaggcgttgccggagctggtcgcgctgggctggacggtaaccgagttcgcggcgggcaagtacgacatcacccggcccaaggcggcaggctgaccccccccactctattgtaaacaagacatttttatcttttatattcaatggcttattttcctgctaattggtaataccatgaaaaataccatgctcagaaaaggcttaacaatattttgaaaaattgcctactgagcgctgccgcacagctccataggccgctttcctggctttgcttccagatgtatgctcttctgctcctgcagcggccgcgaattcctagag

SEQ ID NO: 37 - DNA, 90 bp, Artificial Sequence
  Ptrc1
ttgacaattaatcatccggctcgtataatgtgtggaattgtgagcggataacaatttcacacagctagcattaaagaggagaaatgacat

SEQ ID NO: 38 - DNA, 607 bp, Synechocystis PCC6803
gacgatcccgacttcgttataaaataaacttaacaaatctatacccacctgtagagaagagtccctgaatatcaaaatggtgggataaaaagctcaaaaaggaaagtaggctgtggttccctaggcaacagtcttccctaccccactggaaactaaaaaaacgagaaaagttcgcaccgaacatcaattgcataattttagccctaaaacataagctgaacgaaactggttgtcttcccttcccaatccaggacaatctgagaatcccctgcaacattacttaacaaaaaagcaggaataaaattaacaagatgtaacagacataagtcccatcaccgttgtataaagttaactgtgggattgcaaaagcattcaagcctaggcgctgagctgtttgagcatcccggtggcccttgtcgctgcctccgtgtttctccctggatttatttaggtaatatctctcataaatccccgggtagttaacgaaagttaatggagatcagtaacaataactctagggtcattactttggactccctcagtttatccgggggaattgtgtttaagaaaatcccaactcataaagtcaagtaggagattaattcat

SEQ ID NO: 39 - DNA, 387 bp, Synechocystis PCC6803
taattgtatgcccgactattgcttaaactgactgaccactgaccttaagagtaatggcgtgcaaggcccagtgatcaatttcattatttttcattatttcatctccattgtccctgaaaatcagttgtgtcgcccctctacacagcccagaactatggtaaaggcgcacgaaaaaccgccaggtaaactcttctcaacccccaaaacgccctctgtttacccatggaaaaaacgacaattacaagaaagtaaaacttatgtcatctataagcttcgtgtatattaacttcctgttacaaagctttacaaaactctcattaatcctttagactaagtttagtcagttccaatctgaacatcgacaaatacataaggaattataaccat

SEQ ID NO: 40 - DNA, 591 bp, Synechocystis PCC6803
ataatcagcgggccactctagtgggggaaaaaacctttggtaagggtttgattcaatccttgtttgaactatccgatggggccggcattgccgtcacggtggccaaatacgaaaccccccaacatcacgacatccataaactgggcattatgcccgatgaagtggtggagcaacccctgattagctttgcggaaattacttcccccgccgatgtgcaataccaagccgccttagatttgctcaccggaggagtggcaatcgcccataaatcttcttcaattcccgccatggcaacggctcacaagcccaactaatcaccatttggacaaaacatcaggaattctaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactgacccat

SEQ ID NO: 41 - DNA, 21 bp, Artificial Sequence
  Hom1sll0404_F
cgtggtatctccatagctttg

SEQ ID NO: 42 - DNA, 46 bp, Artificial Sequence
  Hom1sll0404_R
tcccttccccaccactagtccctaaaacaaaaaactgacaataatc

SEQ ID NO: 43 - DNA, 39 bp, Artificial Sequence
  Hom2sll0404_F
ttttgttttagggactagtggtggggaagggaaaagtac

SEQ ID NO: 44 - DNA, 21 bp, Artificial Sequence
  Hom2sll0404_R
gcttacaatcactcattggag

SEQ ID NO: 45 - DNA, 19 bp, Artificial Sequence
  Hom1slr0806_F
gcatcaaaaatggtgcgtc

SEQ ID NO: 46 - DNA, 38 bp, Artificial Sequence
  Hom1slr0806_R
tacttgccttggcactagtgctaagtctggattagtcg

SEQ ID NO: 47 - DNA, 38 bp, Artificial Sequence
  Hom2slr0806_F
atccagacttagcactagtgccaaggcaagtaaagggg

SEQ ID NO: 48 - DNA, 18 bp, Artificial Sequence
  Hom2slr0806_R
ccctctgtggccccgaag

SEQ ID NO: 49 - DNA, 25 bp, Artificial Sequence
  slr0806_IN_F
ccacggctcaaaataacgtctttgc

SEQ ID NO: 50 - DNA, 23 bp, Artificial Sequence
  slr0806_IN_R
ggcacatttgcccttgaatgcgc

SEQ ID NO: 51 - DNA, 23 bp, Artificial Sequence
  sll0404_IN_F
cgtaggggcgtaggaggaacagg

SEQ ID NO: 52 - DNA, 25 bp, Artificial Sequence
  sll0404_IN_R
gaaagcgccgatccatcctatggcc

SEQ ID NO: 53 - DNA, 29 bp, Artificial Sequence
  NdeI_pgp_Syn_F
aaacatatgtggaaaagatcctggaaagc

SEQ ID NO: 54 - DNA, 29 bp, Artificial Sequence
  BamHI_pgp_Syn_R
tttggatccctactgtcgcatcagttgcg

SEQ ID NO: 55 - DNA, 20 bp, Artificial Sequence
  Slr1556-HOM1-F
cctgaatcgttatcggcact

SEQ ID NO: 56 - DNA, 41 bp, Artificial Sequence
  Slr1556-HOM1-R
ggtttgcagagcgtttctagagctaaaatagcggtatcaag

SEQ ID NO: 57 - DNA, 41 bp, Artificial Sequence
  Slr1556-HOM2-F
ataccgctattttagctctagaaacgctctgcaaaccattg

SEQ ID NO: 58 - DNA, 20 bp, Artificial Sequence
  Slr1556-HOM2-R
cccaatccctaccggactat

SEQ ID NO: 59 - DNA, 20 bp, Artificial Sequence
  slr1556_F
aaatttggggtgaagctggg

SEQ ID NO: 60 - DNA, 20 bp, Artificial Sequence
  slr1556_R
tgatgcgacaacaaaaggca

SEQ ID NO: 61 - DNA, 52 bp, Artificial Sequence
  NheI_RBS_NdeI_aceA
aaaagctagcattaaagaggagaaatgacatatgaaaacccgtacacaacaa

SEQ ID NO: 62 - DNA, 42 bp, Artificial Sequence
  aceA _BamHI_AvrII
aaaacctaggggatccttattagaactgcgattcttcagtgg

SEQ ID NO: 63 - DNA, 29 bp, Artificial Sequence
  NdeI-rbcL-7942F
aaaaaacatatgcccaagacgcaatctgc

SEQ ID NO: 64 - DNA, 30 bp, Artificial Sequence
  BamHI-rbcS-7942R
aaaaaaggatccttagtagcggccgggacg

SEQ ID NO: 65 - DNA, 20 bp, Artificial Sequence
  Rbc-HR1-F
ctggaaattctgtcagcggg

SEQ ID NO: 66 - DNA, 62 bp, Artificial Sequence
  Rbc-HR1-R
gtaacgtcgacctgcagactagtgatatccatatgtctagactaggtcagtcctccataaac

SEQ ID NO: 67 - DNA, 65 bp, Artificial Sequence
  Rbc-HR2-F
cctagtctagacatatggatatcactagtctgcaggtcgacgttacagttttggcaattactaaa

SEQ ID NO: 68 - DNA, 20 bp, Artificial Sequence
  Rbc-HR2-R
aaccgtgccaattttcacct

SEQ ID NO: 69 - DNA, 28 bp, Artificial Sequence
  RbcM_Rr_NdeI_F
aaaacatatggaccagtcatctcgttac

SEQ ID NO: 70 - DNA, 28 bp, Artificial Sequence
  RbcM_Rr_SpeI_R
aaaaactagtttacgccggaagggcgct

SEQ ID NO: 71 - DNA, 27 bp, Artificial Sequence
  RbcM_H44N_F
gcggcgaatttcgccgccgagagttcg

SEQ ID NO: 72 - DNA, 24 bp, Artificial Sequence
  RbcM_H44N_R
ggcgaaattcgccgcggtcgccac

SEQ ID NO: 73 - DNA, 33 bp, Artificial Sequence
  RbcX-5UTR-NheI-F
aaaagctagcattaacagcggcttaactaacag

SEQ ID NO: 74 - DNA, 27 bp, Artificial Sequence
  XbaI-cpcBA-F
aaatctagacataaagtcaagtaggag

SEQ ID NO: 75 - DNA, 31 bp, Artificial Sequence
  RbcX-BglII-F
aaaaagatctatgcaaactaagcacatagct

SEQ ID NO: 76 - DNA, 20 bp, Artificial Sequence
  Hom1_sll1031_F
agattttgccccatcaacag

SEQ ID NO: 77 - DNA, 37 bp, Artificial Sequence
  Hom1_sll1031_R
gaacccgattctagataattactagttgaccagcccc

SEQ ID NO: 78 - DNA, 37 bp, Artificial Sequence
  Hom2_sll1031_F
gtcaactagtaattatctagaatcgggttcaaatatg

SEQ ID NO: 79 - DNA, 20 bp, Artificial Sequence
  Hom2_sll1031_R
agtccataccgtcgatgtcc

SEQ ID NO: 80 - DNA, 17 bp, Artificial Sequence
  RbcL_F140I_F
tccgcatccccgtcgcc

SEQ ID NO: 81 - DNA, 17 bp, Artificial Sequence
  RbcL_F140I_R
ggcgacggggatgcgga

SEQ ID NO: 82 - DNA, 21 bp, Artificial Sequence
  RbcL_F345I_F
accttgggcattgttgacttg

SEQ ID NO: 83 - DNA, 21 bp, Artificial Sequence
  RbcL_F345I_R
caagtcaacaatgcccaaggt

SEQ ID NO: 84 - DNA, 1845 bp, Synechococcus elongatus
atgcccaagacgcaatctgccgcaggctataaggccggggtgaaggactacaaactcacctattacacccccgattacacccccaaagacactgacctgctggcggctttccgcttcagccctcagccgggtgtccctgctgacgaagctggtgcggcgatcgcggctgaatcttcgaccggtacctggaccaccgtgtggaccgacttgctgaccgacatggatcggtacaaaggcaagtgctaccacatcgagccggtgcaaggcgaagagaactcctactttgcgttcatcgcttacccgctcgacctgtttgaagaagggtcggtcaccaacatcctgacctcgatcgtcggtaacgtgtttggcttcaaagctatccgttcgctgcgtctggaagacatccgcttccccgtcgccttggtcaaaaccttccaaggtcctccccacggtatccaagtcgagcgcgacctgctgaacaagtacggccgtccgatgctgggttgcacgatcaaaccaaaactcggtctgtcggcgaaaaactacggtcgtgccgtctacgaatgtctgcgcggcggtctggacttcaccaaagacgacgaaaacatcaactcgcagccgttccaacgctggcgcgatcgcttcctgtttgtggctgatgcaatccacaaatcgcaagcagaaaccggtgaaatcaaaggtcactacctgaacgtgaccgcgccgacctgcgaagaaatgatgaaacgggctgagttcgctaaagaactcggcatgccgatcatcatgcatgacttcttgacggctggtttcaccgccaacaccaccttggcaaaatggtgccgcgacaacggcgtcctgctgcacatccaccgtgcaatgcacgcggtgatcgaccgtcagcgtaaccacgggattcacttccgtgtcttggccaagtgtttgcgtctgtccggtggtgaccacctccactccggcaccgtcgtcggcaaactggaaggcgacaaagcttcgaccttgggctttgttgacttgatgcgcgaagaccacatcgaagctgaccgcagccgtggggtcttcttcacccaagattgggcgtcgatgccgggcgtgctgccggttgcttccggtggtatccacgtgtggcacatgcccgcactggtggaaatcttcggtgatgactccgttctccagttcggtggcggcaccttgggtcacccctggggtaatgctcctggtgcaaccgcgaaccgtgttgccttggaagcttgcgtccaagctcggaacgaaggtcgcgacctctaccgtgaaggcggcgacatccttcgtgaagctggcaagtggtcgcctgaactggctgctgccctcgacctctggaaagagatcaagttcgaattcgaaacgatggacaagctctaaggagcctctgactatcgctgggggagtgagcgttgctgcgtaaagctttctccccagcctttcgacttaacctttcaggatttctgaatcatgagcatgaaaactctgcccaaagagcgtcgtttcgagactttctcgtacctgcctcccctcagcgatcgccaaatcgctgcacaaatcgagtacatgatcgagcaaggcttccaccccttgatcgagttcaacgagcactcgaatccggaagagttctactggacgatgtggaagctccccctgtttgactgcaagagccctcagcaagtcctcgatgaagtgcgtgagtgccgcagcgaatacggtgattgctacatccgtgtcgctggcttcgacaacatcaagcagtgccaaaccgtgagcttcatcgttcatcgtcccggccgctactaa

SEQ ID NO: 85 - protein, 472 aa, Synechococcus elongatus
MPKTQSAAGYKAGVKDYKLTYYTPDYTPKDTDLLAAFRFSPQPGVPADEAGAAIAAESSTGTWTTVWTDLLTDMDRYKGKCYHIEPVQGEENSYFAFIAYPLDLFEEGSVTNILTSIVGNVFGFKAIRSLRLEDIRFPVALVKTFQGPPHGIQVERDLLNKYGRPMLGCTIKPKLGLSAKNYGRAVYECLRGGLDFTKDDENINSQPFQRWRDRFLFVADAIHKSQAETGEIKGHYLNVTAPTCEEMMKRAEFAKELGMPIIMHDFLTAGFTANTTLAKWCRDNGVLLHIHRAMHAVIDRQRNHGIHFRVLAKCLRLSGGDHLHSGTVVGKLEGDKASTLGFVDLMREDHIEADRSRGVFFTQDWASMPGVLPVASGGIHVWHMPALVEIFGDDSVLQFGGGTLGHPWGNAPGATANRVALEACVQARNEGRDLYREGGDILREAGKWSPELAAALDLWKEIKFEFETMDKL

SEQ ID NO: 86 - protein, 472 aa, Synechococcus elongatus
MPKTQSAAGYKAGVKDYKLTYYTPDYTPKDTDLLAAFRFSPQPGVPADEAGAAIAAESSTGTWTTVWTDLLTDMDRYKGKCYHIEPVQGEENSYFAFIAYPLDLFEEGSVTNILTSIVGNVFGFKAIRSLRLEDIRIPVALVKTFQGPPHGIQVERDLLNKYGRPMLGCTIKPKLGLSAKNYGRAVYECLRGGLDFTKDDENINSQPFQRWRDRFLFVADAIHKSQAETGEIKGHYLNVTAPTCEEMMKRAEFAKELGMPIIMHDFLTAGFTANTTLAKWCRDNGVLLHIHRAMHAVIDRQRNHGIHFRVLAKCLRLSGGDHLHSGTVVGKLEGDKASTLGFVDLMREDHIEADRSRGVFFTQDWASMPGVLPVASGGIHVWHMPALVEIFGDDSVLQFGGGTLGHPWGNAPGATANRVALEACVQARNEGRDLYREGGDILREAGKWSPELAAALDLWKEIKFEFETMDKL

SEQ ID NO: 87 - protein, 472 aa, Synechococcus elongatus
MPKTQSAAGYKAGVKDYKLTYYTPDYTPKDTDLLAAFRFSPQPGVPADEAGAAIAAESSTGTWTTVWTDLLTDMDRYKGKCYHIEPVQGEENSYFAFIAYPLDLFEEGSVTNILTSIVGNVFGFKAIRSLRLEDIRFPVALVKTFQGPPHGIQVERDLLNKYGRPMLGCTIKPKLGLSAKNYGRAVYECLRGGLDFTKDDENINSQPFQRWRDRFLFVADAIHKSQAETGEIKGHYLNVTAPTCEEMMKRAEFAKELGMPIIMHDFLTAGFTANTTLAKWCRDNGVLLHIHRAMHAVIDRQRNHGIHFRVLAKCLRLSGGDHLHSGTVVGKLEGDKASTLGIVDLMREDHIEADRSRGVFFTQDWASMPGVLPVASGGIHVWHMPALVEIFGDDSVLQFGGGTLGHPWGNAPGATANRVALEACVQARNEGRDLYREGGDILREAGKWSPELAAALDLWKEIKFEFETMDKL

SEQ ID NO: 88 - protein, 472 aa, Synechococcus elongatus
MPKTQSAAGYKAGVKDYKLTYYTPDYTPKDTDLLAAFRFSPQPGVPADEAGAAIAAESSTGTWTTVWTDLLTDMDRYKGKCYHIEPVQGEENSYFAFIAYPLDLFEEGSVTNILTSIVGNVFGFKAIRSLRLEDIRIPVALVKTFQGPPHGIQVERDLLNKYGRPMLGCTIKPKLGLSAKNYGRAVYECLRGGLDFTKDDENINSQPFQRWRDRFLFVADAIHKSQAETGEIKGHYLNVTAPTCEEMMKRAEFAKELGMPIIMHDFLTAGFTANTTLAKWCRDNGVLLHIHRAMHAVIDRQRNHGIHFRVLAKCLRLSGGDHLHSGTVVGKLEGDKASTLGIVDLMREDHIEADRSRGVFFTQDWASMPGVLPVASGGIHVWHMPALVEIFGDDSVLQFGGGTLGHPWGNAPGATANRVALEACVQARNEGRDLYREGGDILREAGKWSPELAAALDLWKEIKFEFETMDKL

SEQ ID NO: 89 - protein, 111 aa, Synechococcus elongatus
MSMKTLPKERRFETFSYLPPLSDRQIAAQIEYMIEQGFHPLIEFNEHSNPEEFYWTMWKLPLFDCKSPQQVLDEVRECRSEYGDCYIRVAGFDNIKQCQTVSFIVHRPGRY

SEQ ID NO: 90 - DNA, 1377 bp, Rhodopseudomonas capsulatus
atggatcagtctaaccgttacgcccggctcgacctcaaggaagccgatctcatcgccggcgggcgccatgtgctctgcgcctatgtcatgaagcccaaggcgggctacggctatctggaaaccgccgcccatttcgccgccgaaagctcgaccgggacgaacgtcgaggtctcgaccaccgacgatttcacccgcggcgtcgatgcgctggtctacgagatcgacccggaaaaggagatcatgaagatcgcctatccggttgaactctttgacagaaacatcatcgacggccgggcgatgctgtgctcgttcctgacgctgacgatcggcaacaaccagggcatgggcgacgtcgaatatgccaagatgcacgaattctacgtgccgccctgctatctgcgcctgttcgacggcccctcgatgaacatcgccgacatgtggcgcgttctgggccgtccggtggtcgatggcggcatggtcgtcggcacgatcatcaagccgaaactgggtctgcgtccgaagccctttgccgatgcctgctatgaattctggctgggcggcgatttcatcaagaatgacgaaccgcagggcaaccagaccttcgcgccgctgaaggaaaccatccgtctcgtcgccgatgcgatgaaacgggcgcaggacgaaaccggcgaagccaagcttttctcggccaacatcaccgccgacgaccattacgaaatggtcgcccgcggcgaatacatccttgaaacttttggcgaaaacgccgatcacgtcgccttcctcgtggatggctatgtcaccggcccggcggcgatcaccaccgcgcgtcggcaattcccgcgccagttcctgcactatcaccgcgcgggccacggcgccgtgacctcgccgcagtcgatgcgcggctacaccgccttcgtgctctcgaagatgtcgcgtctgcagggcgcctcgggcatccacaccggcacgatgggctatggcaagatggagggcgatgcctccgacaagatcatggcctacatgctgaccgacgaggcggcgcagggcccgttctaccatcaggactggctgggcatgaaggccacgacgccgatcatctcgggcggcatgaacgcgctgcgtctgccgggcttcttcgacaacctcggccattcgaacgtgatccagacctcgggcggcggtgcctttggtcacctggatggcggcaccgccggggcgaaatcgctgcgccagtcctgcgacgcctggaaggcgggcgtggatctggtgacctatgcgaaatcgcatcgcgaactggcccgggcgttcgaaagcttcccgaacgatgccgacaagctctatccgggctggcgcgtcgctctgggcgttaactga

SEQ ID NO: 91 - protein, 458 aa, Rhodopseudomonas capsulatus
MDQSNRYARLDLKEADLIAGGRHVLCAYVMKPKAGYGYLETAAHFAAESSTGTNVEVSTTDDFTRGVDALVYEIDPEKEIMKIAYPVELFDRNIIDGRAMLCSFLTLTIGNNQGMGDVEYAKMHEFYVPPCYLRLFDGPSMNIADMWRVLGRPVVDGGMVVGTIIKPKLGLRPKPFADACYEFWLGGDFIKNDEPQGNQTFAPLKETIRLVADAMKRAQDETGEAKLFSANITADDHYEMVARGEYILETFGENADHVAFLVDGYVTGPAAITTARRQFPRQFLHYHRAGHGAVTSPQSMRGYTAFVLSKMSRLQGASGIHTGTMGYGKMEGDASDKIMAYMLTDEAAQGPFYHQDWLGMKATTPIISGGMNALRLPGFFDNLGHSNVIQTSGGGAFGHLDGGTAGAKSLRQSCDAWKAGVDLVTYAKSHRELARAFESFPNDADKLYPGWRVALGVN

SEQ ID NO: 92 - DNA, 1381 bp, Rhodobacter sphaeroides
atggaccagtccaaccgctacgcccggcttgatctgcaggaagccgatctgatcgccggcggccgtcacgttctctgcgcctatgtcatgaagcccaaggcgggctacggctatctggagacggcggcgcatttcgcggccgaaagctccaccggcaccaacgtcgaggtctcgaccaccgacgatttcacccgcggcgtcgatgcgctcgtctatgagatcgacccggagaaggagatcatgaagatcgcctatccggtcgagctcttcgaccgcaacatcatcgacgggcgggcgatgctctgctcgttcctgacgctgacgatcggcaacaaccagggcatgggcgacgtcgaatatgccaagatgcacgatttctatgtgccgccctgctatctgcgcctgttcgacggcccctcgatgaacatcgccgacatgtggcgcgtgctggggcgcgatgtgcgcaacggcggcatggtggtgggcacgatcatcaagccgaagctcgggctgcggccgaaacccttcgcggatgcctgccacgagttctggctgggcggcgacttcatcaagaacgacgagccgcagggcaaccagaccttcgcgccgctgaaggagaccatccgcctcgtggccgatgcgatgaagcgcgcgcaggacgagaccggcgaggccaagctcttctcggccaacatcaccgcggacgaccattacgagatggtggcgcgcggggaatacatcctcgagaccttcggcgagaatgccgaccatgtggccttcctcgtcgacggctatgtgacgggccccgcggccatcaccaccgcgcggcgccagttcccgcgccagttcctgcattatcaccgggcggggcacggcgccgtcacctcgccgcagtcgatgcggggctatacggccttcgtgctctcgaagatggcgcgcctgcagggggcctcgggcatccacaccggcaccatgggctatggcaagatggagggcgaggcggccgacaagatcatggcctacatgctgaccgacgaggcggccgaggggcccttctaccgtcaggactggctggggctgaaggccacgacgcccatcatctcgggcggcatgaacgcgctgcggctgccgggcttcttcgacaatctcggccattccaacgtgatccagacctcgggcggcggcgccttcggccatctcgacggcggcacggcgggggcgaagtcgctgcgccagtcgcacgaagcctggatggcgggggtggatctcgtgacctatgcccgcgagcatcgcgagctcgcccgtgccttcgagagcttcccggcggatgccgacaagttctatccgggctggcgcgaccggctgcatcgcgcggcctgaa

SEQ ID NO: 93 - protein, 459 aa, Rhodobacter sphaeroides
MDQSNRYARLDLQEADLIAGGRHVLCAYVMKPKAGYGYLETAAHFAAESSTGTNVEVSTTDDFTRGVDALVYEIDPEKEIMKIAYPVELFDRNIIDGRAMLCSFLTLTIGNNQGMGDVEYAKMHDFYVPPCYLRLFDGPSMNIADMWRVLGRDVRNGGMVVGTIIKPKLGLRPKPFADACHEFWLGGDFIKNDEPQGNQTFAPLKETIRLVADAMKRAQDETGEAKLFSANITADDHYEMVARGEYILETFGENADHVAFLVDGYVTGPAAITTARRQFPRQFLHYHRAGHGAVTSPQSMRGYTAFVLSKMARLQGASGIHTGTMGYGKMEGEAADKIMAYMLTDEAAEGPFYRQDWLGLKATTPIISGGMNALRLPGFFDNLGHSNVIQTSGGGAFGHLDGGTAGAKSLRQSHEAWMAGVDLVTYAREHRELARAFESFPADADKFYPGWRDRLHRAA

SEQ ID NO: 94 - DNA, 89 bp, Synechocystis PCC6803
ttaacaaaaaagcaggaataaaattaacaagatgtaacagacataagtcccatcaccgttgtataaagttaactgtgggattgcaaaag

SEQ ID NO: 95 - DNA, 1482 bp, Synechococcus PCC7002
atggttttttcgtcagtatccgcaacgcaacaacgagattggccctggattcggcggcgactggtggaaattgtcgggcgggatggcgttgtccggcgcaaggaagaaattcttacctatgaatgtgatggcctcagtgcctaccgtaaacggcctgccctagtggtgctccccagaaccactgcagaaattgcggcgatcgccaaattttgccatgaacaagaaatcccctgggtggcgcggggggctgggacagggctatcggggggcgctttgcccttagaaaatggcattttgattgtgacggcacggatgcgagaaatccttaacattgacctagacaatcagcagatcaccgtgcagccaggggtgattaacaattgggtcacccaagcggtgagtggggctggcttttactatgcacccgatccttcgagccaaacggtctgctctatcggtggcaatgtggcagaaaattccggcggtgtccattgctttaaatatggcgtcaccacgaaccatgtactccaactcaaggtcgtcacccccatgggggatgtggtcacccttggcggcgttgtgccagaaatgccgggctatgacctgatgggcgtctttgtgggatcagaaggaaccctaggtattgccacggaaatcaccttgaaaattttgaagcgaccggaagcggtttgtgtgcttctggcagattatacgtccatcgaggcggcgggaaattcagtagcggcgatcgtcagtgcggggattattccagcgggaatggagattatggacaacttcagcattaatgcggtggaagatatcgtcaagctgaactgttacccccgtgatgcggcggctattttgttgatcgaaattgatgggatggcggcggaggtcaaggcagcaaaggaacgaatcaaggaaatttgcttggcccagggagcgcggcacatcaccagcgccaatgatccagaaacccggctgaaattgtggaaagggcgtaaatcagcctttgcggcggcgggtaatattagtcccgattattttgtgcaggatggcgtgattccccggacaaagttggccgaagttctaaccgagattaatgccattggcgatcgccacgggtacaaaattgccaatgtcttccatgcgggggacggcaacttacacccgttaattttatataacagcgccattccaggggatctagaagccgtagaaaaagtcggcggcgaaatcctcaagctctgtgttgcgaaggggggcagcatttccggagaacatggcatcggcgcggacaaaaaaatgtatatgccggatatgttcaacgaggccgatctcgaaaccatgggctatgtgcgagaagcctttaatccaaagggattagcgaatcccggcaagctatttccaaccccccgcacctgtggcgagtctgccaagatgactaatgtggcagacctaaaaaatagcgaaattttttga

SEQ ID NO: 96 - protein, 493 aa, Synechococcus PCC7002
MVFSSVSATQQRDWPWIRRRLVEIVGRDGVVRRKEEILTYECDGLSAYRKRPALVVLPRTTAEIAAIAKFCHEQEIPWVARGAGTGLSGGALPLENGILIVTARMREILNIDLDNQQITVQPGVINNWVTQAVSGAGFYYAPDPSSQTVCSIGGNVAENSGGVHCFKYGVTTNHVLQLKVVTPMGDVVTLGGVVPEMPGYDLMGVFVGSEGTLGIATEITLKILKRPEAVCVLLADYTSIEAAGNSVAAIVSAGIIPAGMEIMDNFSINAVEDIVKLNCYPRDAAAILLIEIDGMAAEVKAAKERIKEICLAQGARHITSANDPETRLKLWKGRKSAFAAAGNISPDYFVQDGVIPRTKLAEVLTEINAIGDRHGYKIANVFHAGDGNLHPLILYNSAIPGDLEAVEKVGGEILKLCVAKGGSISGEHGIGADKKMYMPDMFNEADLETMGYVREAFNPKGLANPGKLFPTPRTCGESAKMTNVADLKNSEIF

SEQ ID NO: 97 - DNA, 2255 bp, Synechococcus PCC7002
atggttcagaccaaatctgctgggtttaatgccggtgtacaggactaccgcctgacttactacacccccgattacaccccgaaagataccgacttactcgcttgtttccggatgactccccaacctggagtcccccccgaagaatgtgctgcggctgttgcggctgaatcttctaccggtacttggaccactgtatggaccgatggtttaactgacctcgaccgctacaagggtcgttgctacaatgttgaacccgttcccggtgaagacaaccaatatttctgtttcgttgcttaccccctcgatctgtttgaagaaggttctgtaaccaacgttttgacttccttggttggtaacgtattcggttttaaagcgctgcgtgccctgcgcctcgaagatatccgcttccccgttgcgttaatcaaaacttaccaagggcctccccacgggatcactgtagagcgtgacctcctcaacaagtatggtcgtcctctcctcggttgtacgattaagccgaagctcggtctgtctgcgaagaactacggtcgtgcggtttatgaatgtctccgtggtggtcttgacttcaccaaagatgacgaaaacatcaactctcagcctttcatgcgttggcgcgatcgcttcctgttcgttcaagaagctatcgaaaaatcccaagctgaaaccaacgaagttaagggtcactaccttaacgtcaccgctggcacttgcgaagaaatgctcaagcgggctgaattcgctaaggaaatcggcactcccatcatcatgcacgacttcttaactggtggtttcactgcgaatactacccttgcgaagtggtgtcgtgataacggcgttctgctccacatccaccgggcaatgcacgcggtaatcgaccgtcagaagaaccacggtattcacttccgcgttctcgctaagtgtctccgcctctctggtggtgaccacctccactccggtacggttgttggtaagctcgaaggcgatcgcgccgccaccctcggtttcgtagacctgatgcgtgaagactacgttgaagaagatcgttctcgcggtgtattcttcacccaagactacgcttctctccccggcaccatgcctgtggcttccggtggtatccacgtatggcacatgcctgccctcgttgaaatcttcggcgacgattcctgcctccagtttggtggtggtaccctcggtcacccctggggtaacgcacctggtgcaactgcaaaccgtgttgctctggaagcttgtgttcaagctcgtaacgaaggtcgcagcctggcccgtgaaggtaatgatgtcctccgtgaagcaggtaagtggtcgcctgaattggcagccgccctcgacctgtggaaggaaatcaagttcgaattcgataccgttgacactctctaagctcctgatgagcatcagtggatggggaagtttgtcacaatctacctattcactgatgatttcctcccatggagtttaaaaaagttgcgaaggaaacggccatcactttgcaaagctatttgacctaccaagcggtgcgtctaattagtcagcagcttagtgaaaccaatcctggacaggcgatttggctaggagagttctctaaacgtcatccaattcaggaaagtgatctttacctcgaagcgatgatgctagaaaacaaagagctcgtcctcagaatcctgacggtgcgagaaaaccttgcggaaggagttctggagtttttgccagaaatggtcctcagccaaatcaagcagtccaatggaaaccatcgccgttctttattagagcgtttaactcaagttgattcttcatcaactgatcagactgaacctaaccctggtgagtctgatacttcagaagattctgaataaactttgatccgataaagaggacataaatcaatgaaaactttacctaaagaaaagcgttacgaaactctttcttacttgccccccctcagcgaccagcaaatcgctcgccaagtccagtacatgatggatcaaggctatattcctggtatcgagttcgaaaaagatccgactcctgaactccaccactggacactgtggaagctgccccttttcaacgcaagctctgctcaagaagtactcaacgaagtgcgtgagtgccgtagtgaatattctgactgctacatccgtgttgttggtttcgacaacatcaagcagtgccaaaccgttagcttcatcgtttacaagcccaaccaaacccgttactaa

SEQ ID NO: 98 - protein, 471 aa, Synechococcus PCC7002
MVQTKSAGFNAGVQDYRLTYYTPDYTPKDTDLLACFRMTPQPGVPPEECAAAVAAESSTGTWTTVWTDGLTDLDRYKGRCYNVEPVPGEDNQYFCFVAYPLDLFEEGSVTNVLTSLVGNVFGFKALRALRLEDIRFPVALIKTYQGPPHGITVERDLLNKYGRPLLGCTIKPKLGLSAKNYGRAVYECLRGGLDFTKDDENINSQPFMRWRDRFLFVQEAIEKSQAETNEVKGHYLNVTAGTCEEMLKRAEFAKEIGTPIIMHDFLTGGFTANTTLAKWCRDNGVLLHIHRAMHAVIDRQKNHGIHFRVLAKCLRLSGGDHLHSGTVVGKLEGDRAATLGFVDLMREDYVEEDRSRGVFFTQDYASLPGTMPVASGGIHVWHMPALVEIFGDDSCLQFGGGTLGHPWGNAPGATANRVALEACVQARNEGRSLAREGNDVLREAGKWSPELAAALDLWKEIKFEFDTVDTL

SEQ ID NO: 99 - protein, 134 aa, Synechococcus PCC7002
MEFKKVAKETAITLQSYLTYQAVRLISQQLSETNPGQAIWLGEFSKRHPIQESDLYLEAMMLENKELVLRILTVRENLAEGVLEFLPEMVLSQIKQSNGNHRRSLLERLTQVDSSSTDQTEPNPGESDTSEDSE

SEQ ID NO: 100 - protein, 111 aa, Synechococcus PCC7002
MKTLPKEKRYETLSYLPPLSDQQIARQVQYMMDQGYIPGIEFEKDPTPELHHWTLWKLPLFNASSAQEVLNEVRECRSEYSDCYIRVVGFDNIKQCQTVSFIVYKPNQTRY

SEQ ID NO: 101 - DNA, 1461 bp, Synechococcus elongatus PCC 7942
atgaccgccgttgctgcccccaattggactgcgatcgcccaagcttttcgggaagtcctgggtcgagagcaagtcgttgagcggcgcgaagaattactggtttatgaatgtgatggactgaccaatcatcgccaaattccaccgcttgttgttctgccacgtagcaccgaagaagttgccgctgctgtccgtctttgcaatcagtttgatctctcattcgtggcgcggggcgcagggactggcctctcaggtggtgcactaccggttgaagattcggtgctgattgtcactgcacggatgcgtcagattttagagattgactacgacaacctgcgcgttcgcgttcaacccggtgtgatcaatagctgggtgacgcaggcaacgacgggggctggttttttctatgcacctgatccctccagccaaagcgtttgctccattggcggcaatgtcgctgaaaattctggcggagtacattgcctcaagcacggtgtcaccaataaccacgtcctagggctgacactggtgctgccggatgcctccgtgattcaagtcggcggcgcgatcgcagatctacccggctacgacctctgcggcatttttgtcggttcagagggcacgctcggaatcgccaccgaagtgacgctacgactacaaccactcccgcaatccgttcaggtactgcttgccgatttcagcagcattgaagcagccggggctgctgtctcgggcatcatcgcagcgggaattttgccggcgggtttagagctgatggataacttcagcatcaatgccgttgaagatgtggtgaaaagtgattgttatccccgcgatgcggctgccattctgctagcagagttagatggtcgcgcttcggaagttgcgcagcaaattcgcgatgtggaagcagtttgccgacaacatggagcccgatcgatcgcgatcgcaacggatgctgaagatcggctacgactttggaaaggacgtaaggctgcattcgctgctgtcggacggattagcccgagttactacgtgcaagacggtgtaattccgcgatcgacactgccctttgttctgcatgaaattgagcagttaggccaaaaacatggctaccgtgtcgccaacgttttccatgcaggtgatggtaatttacatccgctaattctctacgatcgcaatgatccaggtgctttagagcgtgttgaagcccttgggggtgaaatcctcaagctctgtgtcaacgttggcggcagtatctcgggtgagcatggcattggtgccgataagcgctgctatatgcccgccatgttcagctccgaagatcttgaaacgatgcaatggctacgtcatgccttcgatccgctggaacgcgccaatcccactaaggttttcccaactccccgcacttgtggtgaacgaggatcagttaatagtattccagtcggagttgagctttattaa

SEQ ID NO: 102 - protein, 486 aa, Synechococcus elongatus PCC 7942
MTAVAAPNWTAIAQAFREVLGREQVVERREELLVYECDGLTNHRQIPPLVVLPRSTEEVAAAVRLCNQFDLSFVARGAGTGLSGGALPVEDSVLIVTARMRQILEIDYDNLRVRVQPGVINSWVTQATTGAGFFYAPDPSSQSVCSIGGNVAENSGGVHCLKHGVTNNHVLGLTLVLPDASVIQVGGAIADLPGYDLCGIFVGSEGTLGIATEVTLRLQPLPQSVQVLLADFSSIEAAGAAVSGIIAAGILPAGLELMDNFSINAVEDVVKSDCYPRDAAAILLAELDGRASEVAQQIRDVEAVCRQHGARSIAIATDAEDRLRLWKGRKAAFAAVGRISPSYYVQDGVIPRSTLPFVLHEIEQLGQKHGYRVANVFHAGDGNLHPLILYDRNDPGALERVEALGGEILKLCVNVGGSISGEHGIGADKRCYMPAMFSSEDLETMQWLRHAFDPLERANPTKVFPTPRTCGERGSVNSIPVGVELY

SEQ ID NO: 103 - DNA, 29 bp, Artificial Sequence
  primer
atctcatatgatggaccagtccaaccgct

SEQ ID NO: 104 - DNA, 28 bp, Artificial Sequence
  primer
cgatactagttcaggccgcgcgatgcag

SEQ ID NO: 105 - DNA, 29 bp, Artificial Sequence
  primer
atctcatatgatgcgatgcgcgacatctg

SEQ ID NO: 106 - DNA, 28 bp, Artificial Sequence
  primer
acatactagtttacgccgcctgcggctt

SEQ ID NO: 107 - DNA, 20 bp, Artificial Sequence
  primer
tgtgctacttacccttgtcc

SEQ ID NO: 108 - DNA, 61 bp, Artificial Sequence
  tcatctcccagcacttttggtctagattttttactagttttcaggaaagcacagtggtttc
tcatctcccagcacttttggtctagattttttactagttttcaggaaagcacagtggtttc

SEQ ID NO: 109 - DNA, 50 bp, Artificial Sequence
  primer
gctttcctgaaaactagtaaaaaatctagaccaaaagtgctgggagatga

SEQ ID NO: 110 - DNA, 20 bp, Artificial Sequence
  ptimer
ttacggttcgctcccattag

SEQ ID NO: 111 - DNA, 20 bp, Artificial Sequence
  primer
tttctcccgttgcattggcg

SEQ ID NO: 112 - DNA, 61 bp, Artificial Sequence
  primer
cgtgactgaaattccagctctctagattttttactagttttgacacagacgaactgttgcc

SEQ ID NO: 113 - DNA, 50 bp, Artificial Sequence
  primer
gtctgtgtcaaaactagtaaaaaatctagagagctggaatttcagtcacg

SEQ ID NO: 114 - DNA, 20 bp, Artificial Sequence
  primer
gtcaatcatctttgcggatc

SEQ ID NO: 115 - DNA, 18 bp, Artificial Sequence
  primer
cgagatccatgccggcgc

SEQ ID NO: 116 - DNA, 60 bp, Artificial Sequence
  primer
aaccctcgagctgcagactagtgatatccatatgtctagagcggttttcctccagcaaaa

SEQ ID NO: 117 - DNA, 60 bp, Artificial Sequence
  primer
gctctagacatatggatatcactagtctgcagctcgagggttttgttggtttttgtgacc

SEQ ID NO: 118 - DNA, 19 bp, Artificial Sequence
  primer
gcggctttctcacccatgg

SEQ ID NO: 119 - DNA, 20 bp, Artificial Sequence
  primer
gacagctcgtcagtttgagc

SEQ ID NO: 120 - DNA, 60 bp, Artificial Sequence
  primer
ggctctcgagctgcagactagtgatatccatatgtctagagtcgtctctccctagagata

SEQ ID NO: 121 - DNA, 60 bp, Artificial Sequence
  primer
gactctagacatatggatatcactagtctgcagctcgagagcctgatttgtcttgatagc

SEQ ID NO: 122 - DNA, 19 bp, Artificial Sequence
  primer
agatcagcgatcgctcgca